SEQUENCE LISTING (57 sequences)

SEQ ID NO: 1 - DNA, 894 bp, Homo sapiens
cgggccctgcgggcgcggggctgaaggcggaaccacgacgggcagagagcacggagccgggaagcccctgggcgcccgtcggagggctatggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgtaattcctagctgtcgtgggatggagggaagggcggctgggagcagagcaggggcctggggtggggcaggtgctgctggttcaggaataggaagaggggatagggaggagggagccttggccctgtgatgggtgggccccacttcaggcaaacttagatggcaaaagagcaatctggatccgccttagccagatacataagggtatttgccttcactttcagccagcattccccccagcgatcctagccagatattacagatg

SEQ ID NO: 2 - DNA, 1503 bp, Homo sapiens
cgggccctgcgggcgcggggctgaaggcggaaccacgacgggcagagagcacggagccgggaagcccctgggcgcccgtcggagggctatggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgagcaccctggggagctgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtgacacggcgcccacttgccacctaggcgctctggtggcccttgcagaagccctaagtacggttacttatgcgtgtagacattttatgtcacttattaagccgctggcacggccctgcgtagcagcaccagccggccccacccctgctcgcccctatcgctccagccaaggcgaagaagcacgaacgaatgtcgagagggggtgaagacatttctcaacttctcggccggagtttggctgagatcgcggtattaaatctgtgaaagaaaacaaaacaaaacaaaaaaaaaaaaaaaaaa

SEQ ID NO: 3 - DNA, 1527 bp, Homo sapiens
cgggccctgcgggcgcggggctgaaggcggaaccacgacgggcagagagcacggagccgggaagcccctgggcgcccgtcggagggctatggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgagcaccctggggagctgtccagagcgctgtgccgctgtctgtggctggaggcagaatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtgacacggcgcccacttgccacctaggcgctctggtggcccttgcagaagccctaagtacggttacttatgcgtgtagacattttatgtcacttattaagccgctggcacggccctgcgtagcagcaccagccggccccacccctgctcgcccctatcgctccagccaaggcgaagaagcacgaacgaatgtcgagagggggtgaagacatttctcaacttctcggccggagtttggctgagatcgcggtattaaatctgtgaaagaaaacaaaacaaaacaaaaaaaaaaaaaaaaaa

SEQ ID NO: 4 - DNA, 1665 bp, Homo sapiens
cgggccctgcgggcgcggggctgaaggcggaaccacgacgggcagagagcacggagccgggaagcccctgggcgcccgtcggagggctatggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgccacccccgtcccttgcaggagcaccctggggagctgtccagagcgctgtgccgctgtctgtggctggaggcagagtaggtgtgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtgacacggcgcccacttgccacctaggcgctctggtggcccttgcagaagccctaagtacggttacttatgcgtgtagacattttatgtcacttattaagccgctggcacggccctgcgtagcagcaccagccggccccacccctgctcgcccctatcgctccagccaaggcgaagaagcacgaacgaatgtcgagagggggtgaagacatttctcaacttctcggccggagtttggctgagatcgcggtattaaatctgtgaaagaaaacaaaacaaaacaaaaaaaaaaaaaaaaaa

SEQ ID NO: 5 - DNA, 1638 bp, Homo sapiens
cgggccctgcgggcgcggggctgaaggcggaaccacgacgggcagagagcacggagccgggaagcccctgggcgcccgtcggagggctatggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgagcaccctggggagctgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtgacacggcgcccacttgccacctaggcgctctggtggcccttgcagaagccctaagtacggttacttatgcgtgtagacattttatgtcacttattaagccgctggcacggccctgcgtagcagcaccagccggccccacccctgctcgcccctatcgctccagccaaggcgaagaagcacgaacgaatgtcgagagggggtgaagacatttctcaacttctcggccggagtttggctgagatcgcggtattaaatctgtgaaagaaaacaaaacaaaacaaaaaaaaaaaaaaaaaa

SEQ ID NO: 6 - DNA, 1089 bp, Homo sapiens
cgggccctgcgggcgcggggctgaaggcggaaccacgacgggcagagagcacggagccgggaagcccctgggcgcccgtcggagggctatggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtgacacggcgcccacttgccacctaggcgctctggtggcccttgcagaagccctaagtacggttacttatgcgtgtagacattttatgtcacttattaagccgctggcacggccctgcgtagcagcaccagccggccccacccctgctcgcccctatcgctccagccaaggcgaagaagcacgaacgaatgtcgagagggggtgaagacatttctcaacttctcggccggagtttggctgagatcgcggtattaaatctgtgaaagaaaacaaaacaaaacaaaaaaaaaaaaaaaaaa

SEQ ID NO: 7 - DNA, 511 bp, Homo sapiens
gcctcccaagtagctgggactacaggagcccaccaccacccccggctaattttttgtatttttagtagagacggggtttcaccgtgttagccaagatggtcttgatcacctgacctcgtgatccacccgccttggcctcccaaagtgctgggattacaggcatgagccaccgcgcccggcctccattcaagtctttattgaatatctgctatgttctacacactgttctaggtgctggggatgcaacaggggacaraataggcaaaatccctgtccttttggggttgacattctagtgactcttcatgtagtctagaagaagctcagtgaatagtgtctgtggttgttaccagggacacaatgacaggaacattcttgggtagagtgagaggcctggggagggaagggtctctaggatggagcagatgctgggcagtcttagggagcccctcctggcatgcaccccctcatccctcaggccacccccgtcccttgcaggagcaccctgggg

SEQ ID NO: 8 - DNA, 601 bp, Homo sapiens
gggaatgctggctgaaagtgaaggcaaatacccttatgtatctggctaaggcggatccagattgctcttttgccatctaagtttgcctgaagtggggcccacccatcacagggccaaggctccctcctccctatcccctcttcctattcctgaaccagcagcacctgccccaccccaggcccctgctctgctcccagccgcccttccctccatcccacgacagctaggaattacgtggggcaggacacgcagccatcgccatgttcatagaagccaggcaggcaggtcccacagtcagtayctctgcgggaacctgcaatccaggagaggcatgcgtcaccatgggacaggagtgggtcaggcagggagaagggggtctgggagtagagagccctgggtgggggtactcacagagtagccgtgtgtggcggtgcagggccccgcagtctaggcatggttggcagtagaagggtgaactgctgacacattggctgacctggcactccacaaaccagcctggcttacagccacagcgggtgtcggccactgctgaacagttctccagcgccacctgggaggctggtgggggtgcagggagatg

SEQ ID NO: 9 - DNA, 511 bp, Homo sapiens
acgccgggctagtttttgtattttaagtagagacggcatttcaccatattggtcaggctggtctcgaactcctgaccccaagtgttccgcccgcctctgcctcccatagtgctagaattacaggcctgagctactgcgcttggccccttgcggtacttttggcccaacctcctccatggctggggacgcggaggccgagagagaagtcacttgccctggctctaccttgaagtggttctcagggttggggcgagastcggggtggggaccgagatgcagctctatcctgtgcccctggtcgcagcaggcagcccagcgcttcgcgtgttctacttggcctgtccgctgccgcctaatgagctcaggtctaggccgagcagagggggcacctggtcggactcggttgggctcgggcggccccgcctccccccgcccgccaggcgggcccttctcgacggcgcggggcgggccctgcgggcgcggggctgaaggcggaaccacgacgggcaga

SEQ ID NO: 10 - DNA, 401 bp, Homo sapiens
gcacacaatcttgtccacgagcaggggtggcctgatgaccctggtcctctctgccttcttcactgctttggtcaccaacagcagatccgtgaagaggaagcagtacacatccatctgcagtggcaggagggggggtggccagagaggccagcagggtcagggccagggactgtggcaccagcctcccctgggagcactcgstttttgtcaaacactcctcccaccatgaaccttcaagtaaaattaaaatgaagaggaagatgattctctttcactgagatgtacgttgatgacttatctcaatacaagtttacattaaaaagaagactggtgactttgaagtcaccgtgtgcaagctctcagcctgccccatgcctgaaggggccatggcacagtagcaa

SEQ ID NO: 11 - DNA, 601 bp, Homo sapiens
ctctttcactgagatgtacgttgatgacttatctcaatacaagtttacattaaaaagaagactggtgactttgaagtcaccgtgtgcaagctctcagcctgccccatgcctgaaggggccatggcacagtagcaaggctctggggttcctggcccacttgtggaatatggctgccctcccaccaggaacccatgtggcaccaacaaatgggcacaactggtgccttcaagggctaggaccctactgctggatttgtgaatggcaggcagagaactgtcattcactatgacaaggtcactgrttcttgagtaaccaccgaagggactgcagagctgagaaccagtgactggggcccaggaggtccctaggaagggcagggcacaggggtggggtggccctggaatcaccttgctgtccttcccctccttcatcctcaggctcccctccagcagcagctgccgcgtctcctccggggaggcgccagggatgggcgctgtcaagtccaggtgcagaaattccttcaggagctggggacggatggcgtgaacgtaggggaggccagagactgactcccatctcagctaggcatcctcagtgagat

SEQ ID NO: 12 - DNA, 601 bp, Homo sapiens
cctggacctgaagctccctcacctgcccctagatcggctccgtggagcgcttcatccaccacgtgaacgcgtgcatgcggcagcggcaggagcggcagcggctggcggccgtggtgagccgcatcgacgcctacgaggtggtggaaagcagcagcgacgaagtggacaaggtgggcgtgtgtgtctgcgggggttgccggctgggagagaggaaggcccagctggcaggcaggggctgtgctgggcatctcactgaggatgcctagctgagatgggagtcagtctctggcctcccctacgytcacgccatccgtccccagctcctgaaggaatttctgcacctggacttgacagcgcccatccctggcgcctccccggaggagacgcggcagctgctgctggaggggagcctgaggatgaaggaggggaaggacagcaaggtgattccagggccaccccacccctgtgccctgcccttcctagggacctcctgggccccagtcactggttctcagctctgcagtcccttcggtggttactcaagaaccagtgaccttgtcatagtgaatgacagttctctgcctgccattcacaaatccag

SEQ ID NO: 13 - DNA, 401 bp, Homo sapiens
ctgcgtgggcagacccgtgctggtgtgcacgccagtgtgagggagtgcggtggggtctgtgtacttgtgcattttaatcatgtatgcaggagccaggcctgcgtgtgggcaggcgtgtgacaggtctgccgtggtatgcagatgcgcctgtgtgagcacctctgcgggcaggtgtctgtggacctgcgtgtgatcccatgygggcttcccttttccgttgggaaggtgggggcagctggggctgtgggtggggcgggggctcgtgggggcggggcccaagctgaagcgactggccgcctggttctaacacacatgcctcctgtcctgggggcagggaggtgctgctgcctgtatttgaaaggaagggcattgcgctgggcaaagtggacatctacctggac

SEQ ID NO: 14 - DNA, 601 bp, Homo sapiens
ccccaaatttcatagctaaaccgttgtccctgtggaatgccccctactcccggccccaagaaaacccctggttataattacatttgaacaaagaaaattaggaactcggtggcagaggactttcatataatgatatttgctctcctcctatagtaagaaggctccaaagaaggttttatcttcttttgtgtaatccaccaaagagatgtcactgacgttcaccattagcttgtccccttcttgcaaggagaacatggctccgaggtagatgggctggaaccagttgctacctacttcgcayacagacttggtccccatgaggagctgggttggctcagggtagctgtctgttaccttggtgatgaccacagtgatggagtctggcttgtttggtcggcctgcttgtctgatttcactgcactcagaggtcatcccacggaatgtgacctgggagtaaatgaagtagtctcccgactctgggatcagcaggaatttgttggtatagttcattcggttcttggtgaaggccaggcctagttcatgttcccagtgcagagctgggaactgatttttaaagtgctgtgtgggagtttgtctcaca

SEQ ID NO: 15 - DNA, 801 bp, Homo sapiens
gtagatgggctggaaccagttgctacctacttcgcatacagacttggtccccatgaggagctgggttggctcagggtagctgtctgttaccttggtgatgaccacagtgatggagtctggcttgtttggtcggcctgcttgtctgatttcactgcactcagaggtcatcccacggaatgtgacctgggagtaaatgaagtagtctcccgactctgggatcagcaggaatttgttggtatagttcattcggttcttggtgaaggccaggcctagttcatgttcccagtgcagagctgggaactgatttttaaagtgctgtgtgggagtttgtctcacaactggaaagacaagaaagaggattaattttctcattgggaaactgtagactttgcttaaaaagygtctcatatcattttcaaaatagactaaagtgatcgaatatacctaacagctaaaaactgctttggggaggaatgaatgaagaatatgtgactggacatacacatttgttcaaagagaataacatcttggactagtgacctggggcaaattactttgcctttctgagactgagtggtctgacctgaaaatttttaattttacctgagacctatgatcctggagaacaatgaaggttaaggaatccccctattcttctgtgtttaggaaaatggcttgctgcaagaattatcctttcccaaatgactcagataaaactcatcgatgcctctcttgtttacgtatgacaaggccaggcaccagacccttcaaattcccattctttgccccataagtgattgg

SEQ ID NO: 16 - DNA, 601 bp, Homo sapiens
tcttctgcaaaaccaagggctgtcatttctattttgcaattttctcatggtgtttcaattccaggccttggcatgctgctctcctggattctttcatcagcccctggcagggtctttgtacttcagttttctccttttccttgatctatttcctggcctgccttcagagggctttttctaaagcatcactttatctagatactacactgcttataaaccttcaatggctcctcatggctcttaagacccagtccaatctcattttgtcttggcattcaaagtcctaacttatcccagtctygctatccattatttacttctctctaagccctctgtgttcccagccatgagaggcaaccactgtgaattgattcaaggtatctgaagaaacagagttaagtgtagttactttattgaattagtagggacacagagacctggcccagaaatgggagaatcagaagaaattgctcctgaacagagtcttagaggtcctgcaggtactagccaggtgataaaagaggtaatgactattccagacaaggccaacagcttgcacaacagcaaatacctcaataccctgcatctgtgatgtggtgtc

SEQ ID NO: 17 - DNA, 601 bp, Homo sapiens
cctacaacagaaaccaagtttggtttgaggaaaaaaaaaaaaaaaaaaaaaagaagaagaaggagaagaagaaagctttcccaagcatatttatatacagtatgctcatgtgctccttccttcgtttacagaaggaagttaggaaagtccctgaaggaggagagaaagaattcatcaagtcagtgggtggggcaaattaaaatatacctgttccctgcactggaggcttaccagctgtgccagtctggggagtgtgcttctggaagtgaaagtgagggatgagaggtgtgtggtttgcagrttgggaaacggaaatcacatttgcatcagctctttgcaaagtgctgcctagccctctgtcattttgaacctcatagaaattcattctcagtgtacagatgggaatagcaaagttctaaaaggtgaaggcacttgtcctaggtcatccaaggatgaagacagaggagctaggaagatgacctagttctaaatcacggcttggagttgtaacctctagcacatgactgcccatgaaaggaaagtatttccagtctgcattgaccattgtttaatcagagtatgaggccacagatcgaggtga

SEQ ID NO: 18 - DNA, 682 bp, Homo sapiens
atgagaggtgtgtggtttgcagattgggaaacggaaatcacatttgcatcagctctttgcaaagtgctgcctagccctctgtcattttgaacctcatagaaattcattctcagtgtacagatgggaatagcaaagttctaaaaggtgaaggcacttgtcctaggtcatccaaggatgaagacagaggagctaggaagatgacctagttctaaatcacggcttggagttgtaacctctagcacatgactgcccatgaaaggaaagtatttccagtctgcattgaccattgtttaatcagagtaygaggccacagatcgaggtgactgtctgtgagggtagaacattaaccactactccctgattagtctaaagttaattgatcatgtgatgtgctttgcctgcagttgggtgtgggggccacaacatgtaataaaagattatatttattaagtgcttactttgtgccaatcactgctctaagttaaatacatcaataaaattattcaatcctgagataaattttgtgacaataaagctatcattctcattttacacataagaaaataaagcacagacggcaagtggtagagccaggattgggactcaggcaggctggctgctggcttcttcaccatcacacttcaccataggtactgctggatcatgcatgttcataaatacc

SEQ ID NO: 19 - DNA, 606 bp, Homo sapiens
aactgtctgaaggtcagccccctctccactcctgaaaaggcataattcagagttggcatttttagttttgcttttctatttccaaatatgaaacagacaaggagtcaggagacccatcacgaacttgctatgtgacttcaggctgttcactcccctgtctctggtctccaggaaaacaagagggctgagaatccacccagatccatggtttttgtgtatctggggagtgggctattccattgaaatgtgtgttttgatgatcatggctaagtgggacttyagtgactcaaaccctgtgttcagatgaagcctgctcagatttctcctataagcgtagaagaaatgagggttttggaggcccaggctggggtctcactggacagtcttgagaggtgggagtgaatgtgaactctggagcacattggtttccacgtgtccctctgttgtgtaaccccaggcaaatcatggagcctccttaggcctcagagtcctcatggactacaacaggatgacagaactacttgttatgaagaataagtgacatgatgctcataaagtcctgggtacattctgtaattcacgttcttgattactgcccttcattgg

SEQ ID NO: 20 - DNA, 3070 bp, Homo sapiens
ggcgccgtcttgatactttcagaaagaatgcattccctgtaaaaaaaaaaaaaaaatactgagagagggagagagagagagaagaagagagagagacggagggagagcgagacagagcgagcaacgcaatctgaccgagcaggtcgtacgccgccgcctcctcctcctctctgctcttcgctacccaggtgacccgaggagggactccgcctccgagcggctgaggaccccggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacagaaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaagttgtaggcgaatcatttgttcaaagctgttggcctctgcaaaggaaataccagttctgggcaatcagtgttaccgttcaccagttgccgttgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggcctaaggtggttgtgctcggagggtttcttgtttcttttccattttgtttttggatgatatttattaaatagcttctaagagtccggcggcatctgtcttgtccctattcctgcagcctgtgctgagggtagcagtgtatgagctaccagcgtgcatgtcagcgaccctggcccgacaggccacgtcctgcaatcggcccggctgcctcttcgccctgtcgtgttctgtgttagtgatcactgcctttaatacagtctgttggaataatattataagcataataataaagtgaaaatattttaaaactacaa

SEQ ID NO: 21 - DNA, 3067 bp, Homo sapiens
ggcgccgtcttgatactttcagaaagaatgcattccctgtaaaaaaaaaaaaaaaatactgagagagggagagagagagagaagaagagagagagacggagggagagcgagacagagcgagcaacgcaatctgaccgagcaggtcgtacgccgccgcctcctcctcctctctgctcttcgctacccaggtgacccgaggagggactccgcctccgagcggctgaggaccccggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaagttgtaggcgaatcatttgttcaaagctgttggcctctgcaaaggaaataccagttctgggcaatcagtgttaccgttcaccagttgccgttgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggcctaaggtggttgtgctcggagggtttcttgtttcttttccattttgtttttggatgatatttattaaatagcttctaagagtccggcggcatctgtcttgtccctattcctgcagcctgtgctgagggtagcagtgtatgagctaccagcgtgcatgtcagcgaccctggcccgacaggccacgtcctgcaatcggcccggctgcctcttcgccctgtcgtgttctgtgttagtgatcactgcctttaatacagtctgttggaataatattataagcataataataaagtgaaaatattttaaaactacaa

SEQ ID NO: 22 - DNA, 516 bp, Homo sapiens
ttctcctcccccaggcccagctcgccagggcctcgggccgtgctgcagtttctggcctttggtgtcgctccccgccccccagccccgcagaatcccggcttcttttctgtctgcgcggccgggaccgcccaggcaggcgccggggctccggggctccggggggagggactcggcggctcggctcggctccgcttctttctyctgcctgcaaatatttgctgcctcgctggaaatccgacgatttcgcgcgcgctctgcttgcaaagtctttaagtaaacacgctcaaatgaccgccccgggcggcccgaggcacgctctctccccctccgcgggattagtaactttaggacttcgaccccggggctccgctttgcctgttacccaggtcgggcagcgcgcgggcgcccggggccgcttctcccggcacctcggccccgcggagctcgcctggaagcgccggttgcctggctctggtgggtggcccggccgcgagcatctccgcgccctgggtctgt

SEQ ID NO: 23 - DNA, 601 bp, Homo sapiens
ccttttcccagagctagtgcctttggtttttagacaggtctcttacctcctggcttcaggaatggaattctgatgctgaaggggtttgggggggaagacccctgtcttaagtttgagggatctgagatttcccagattcccggctgcacagaattttcttgcgtctttgctttcaagtcgcctccttgcctgcaactcttgccttactctgtctctgggtactgccctccattagcctcccacctaagatgtaggatacacccccgtttaaataaaggcaattccagtaccacctctttcyccctttcacctggagaagttcaggagagttctgaaatgtaaaaaaagaagaccaggcctgtgtagtttgaggaaaaaagatcgaacactttccagctctaagtttgttcctaaagaagaaacaggggtaaaacatcgggcagaaaaagtgtggggctttctgagtccagccagaccgaattctgccctgattcccctactcagagcctgccttgacacggatgacatagcccctccggcagcaggcgtcctctaccctgctgtcgccaggttttaaacaatcgtttctgcggatggggcg

SEQ ID NO: 24 - DNA, 601 bp, Homo sapiens
ccttactggtagtagttcaaagagaaaagaagaccaaatgccaggtatgagaggactcacacgacgtttcccataggaattgcctgtggaaagttctcaaaggctgagaccaagaaaggaggaaagtggcattaggctcactggttgctggtgaagcctctgaaattcatttaatcagctccaaccattttgaagcattagcataacaaatccatccattgcactgagtctgtaatccttcctgggaagccacacgccacaacttcaggaacccattctggatggtttctggaacattccrtcctcatactaaatgtctctatctgtcaaacggtgctcatcaatgcacttaaaccagctgctctggtcctgttcataccattattctattcaggaatacactgcagatttgataaccaagaattcagtctccttaaaatgttcacaagcgacttaagaaacatggatggggaaaaagaggggaaaagagctgttctgtgtatcaaatttgaaaaaaaaaaaaaaagaagaagaagaaagaaagaaaaaggaaaaaaaaaaaaactgtcccaagccagctgacacgattggaggctatcct

SEQ ID NO: 25 - DNA, 601 bp, Homo sapiens
ttataaggaaaaaaaaaaaatcttccttttggaaaacaaaaaaagccaccggtcctattttgttgtttccttacattttaaactctttgcagaaagagagaatgaaagagaaaggtaaatagaattgtaatgtgtggcagggggtctggaaactaagaggacccatttggttactgagaggtaaaaagtcttggctaaatctgtgctacaaatttgaaatggatcctctgtataaaagacatggaaaaaaatgtctgacagatttcgtctgatgtctttgtccgaaccaactttcacgtgyagagagctagctcctgaggaaggtggctggcctgggggatgttaccataccagggaaaataaactgtcattttctccttttatatcatgttagcagagcggtaattgccagtcacctgaatttagttgtgtcatttctgatctttccatttggggtcctctcattttgtaatatatcttaatcttaaaaaatttttttttcagttttttttttttcctgaaatcattctggctcagaggccatgattctagtgaggcttcaagtttacattaactctaaagacaaaaatggttattttgg

SEQ ID NO: 26 - DNA, 511 bp, Homo sapiens
tctggaaactaagaggacccatttggttactgagaggtaaaaagtcttggctaaatctgtgctacaaatttgaaatggatcctctgtataaaagacatggaaaaaaatgtctgacagatttcgtctgatgtctttgtccgaaccaactttcacgtgcagagagctagctcctgaggaaggtggctggcctgggggatgttaccataccagggaaaataaactgtcattttctccttttatatcatgttagcagagyggtaattgccagtcacctgaatttagttgtgtcatttctgatctttccatttggggtcctctcattttgtaatatatcttaatcttaaaaaatttttttttcagttttttttttttcctgaaatcattctggctcagaggccatgattctagtgaggcttcaagtttacattaactctaaagacaaaaatggttattttggtctttctctgtgttgctctctctctccttcctttttagctctttctgattctcc

SEQ ID NO: 27 - DNA, 511 bp, Homo sapiens
gtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaaygcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaagg

SEQ ID NO: 28 - DNA, 701 bp, Homo sapiens
gaaaacgtctgaacaatggcatataatcttatccatagaacaaaaattctagccattatctaaatcagcagagcaacacaccaacaccttttgcccttcaaaatgaagcaaacttctggaaaacatcttcatttttctttgtaccctctagctcaagcagatttgacatcatcaggtttgacttgtccatacgctttgcaragagcccaggaaggggaaaaccctggaattctgaggcattcattgatcatttgaacatgctgtgactattgtcctaggagtctggaaactagggcttcattcctggctgtgctgccatttaacaggatgatgtgaggctctctgtttgatttctctgggagcacacttcttatctatgtagttagatgagatactctctaagacctactggagattttttttttttttttaagaacaactcttctgaggaaaagattcttctcacccccagccatatttttatttatttattttttctgagatggagtctcattgtgttgcccaggctggagtacagtggcacaatctcagctcactgcaacctccgcctcccgggttcaagcaattctcgtgcctcagcctccagaatagctgggattacaggcgcccaccaccatgcccagctaatttttgtatctttagtagagatggagtttcaccatattggccaggctggtctc

SEQ ID NO: 29 - DNA, 601 bp, Homo sapiens; identical to SEQ ID NO: 14

SEQ ID NO: 30 - DNA, 601 bp, Homo sapiens; identical to SEQ ID NO: 16

SEQ ID NO: 31 - DNA, 601 bp, Homo sapiens; identical to SEQ ID NO: 17

SEQ ID NO: 32 - DNA, 682 bp, Homo sapiens; identical to SEQ ID NO: 18

SEQ ID NO: 33 - DNA, 606 bp, Homo sapiens; identical to SEQ ID NO: 19

SEQ ID NO: 34 - DNA, 801 bp, Homo sapiens
agaatgctttgctcaaacacctggacactcaatacagtgaccaaaacagattcaacctgtgctttgaagaaagagactttgtcccaggagaaaaccgcattgccaatatccaggatgccatctggaacagtagaaagatcgtttgtcttgtgagcagacacttccttagagatggctggtgccttgaagccttcagttatgcccagggcaggtgcttatctgaccttaacagtgctctcatcatggtggtggttgggtccttgtcccagtaccagttgatgaaacatcaatccatcagaggctttgtacagaaacagcagtatttgaggtggcctgaggatttccaggatgttggctggtttcttcataaactctctcaacagatactaaagaaagaaaargaaaagaagaaagacaataacattccgttgcaaactgtagcaaccatctcctaatcaaaggagcaatttccaacttatctcaagccacaaataactcttcactttgtatttgcaccaagttatcattttggggtcctctctggaggttttttttttctttttgctactatgaaaacaacataaatctctcaattttcgtatcaacaccatgttctgtctcactaacctccaaatggaaaataatagatctagaaaattgcaactgcccttagaggtttccagtctccattgattttctttcagatccaataataccgttctgtcctgctgtgttgattatggaatgtatcctaatcatgggaagggcaccttgggagaagttgcagatggctgacgtgct

SEQ ID NO: 35 - DNA, 201 bp, Homo sapiens
  misc_feature (30)..(30): n is a, c, g, or t
ttgtgaatgtgaacttagcacttttatcanttggcttaatcacaccantgtcactatagctgggcctcctgcagacatatattgtgtgtaccctgactcgytctctggggtttccctcttctctctttccacggaaggttgtgatgaagaggaagtcttaaagtccctaaagttctcccttttcnttgtatgcactgtcac

SEQ ID NO: 36 - DNA, 601 bp, Homo sapiens
agctctgttgggatgtttttgagggactttctcatcttcaagttctgtatttgaatcataactatcttaattcccttccaccaggagtatttagccatctgactgcattaaggggactaagcctcaactccaacaggctgacagttctttctcacaatgatttacctgctaatttagagatcctggacatatccaggaaccagctcctagctcctaatcctgatgtatttgtatcacttagtgtcttggatataactcataacaagttcatttgtgaatgtgaacttagcacttttatcarttggcttaatcacaccaatgtcactatagctgggcctcctgcagacatatattgtgtgtaccctgactcgttctctggggtttccctcttctctctttccacggaaggttgtgatgaagaggaagtcttaaagtccctaaagttctcccttttcattgtatgcactgtcactctgactctgttcctcatgaccatcctcacagtcacaaagttccggggcttctgttttatctgttataagacagcccagagactggtgttcaaggaccatccccagggcacagaacctgatatgtacaa

SEQ ID NO: 37 - DNA, 401 bp, Homo sapiens
ataagattgcagatgaagcattttacggacttgacaacctccaagttctcaatttgtcatataaccttctgggggaactttacagttcgaatttctatggactacctaaggtagcctacattgatttgcaaaagaatcacattgcaataattcaagaccaaacattcaaattcctggaaaaattacagaccttggatctcygagacaatgctcttacaaccattcattttattccaagcatacccgatatcttcttgagtggcaataaactagtgactttgccaaagatcaaccttacagcgaacctcatccacttatcagaaaacaggctagaaaatctagatattctctactttctcctacgggtacctcatctccagattctcattttaaatcaaaat

SEQ ID NO: 38 - DNA, 1314 bp, Homo sapiens
ccagcggggtgacgcaactgcaggaggggtctgtattggtagtgatgaggctgtggtgctggcttctagggccattcccgtctgtatcatttagacaccatggttcagtgtctggaatcctcacagacttagtcctaggccctcctctcatctcacactgtctccactttgggatcttacctatgcccacagcttcaatcaccacccatacaaacaatacaaacctgacgcccagactgacatctccagcttccacctctgttctgagctccagacctatgaagccacctcttatttgaccttcaccttggatgactcacagggacctcaaactcaatatgttcaagacgaaatttttgcttttcccccataacttggttggcttccactgcttttttctgcagcgaatggcaccctcaacatcgcattgtggccaaatgctcaggcaacagcctttagagctttctcttccttatccatgttgaattctttacaaaattctgcccattttacttcctatatattctcagacatcccacatctctccatctctaccattattagtcaagctacctgttgcaaagaccactttagctccctaattcctaactggtgttcagcgtccgctttgtttatacctccttatcacccatatgtcatttatagtcagatagaatgatcttttcaaaatacagatctgatcatgtatctttcctgctaaccagccttcagtggtgtctcctcactgtcaaagtgaatattaaaaatctagatacctcatacatggttctacatgcctggtcacatgctgtctgtagttccatctcacttctttccccaaactctctgcactccagccacagaccttcttctagttttcccagcgccacgaggcctttgaacaagctggttcctgcacctaaaaagcttcttagtcacatttttctcatccttcagctctcagcctaaacatctttgctacacaggagtcttccctaacctctagaattgctactcaaagtgtggttcatggaccagcagcattcctgggagcttgttagaaatgcagaatccaagcctcacccagacccttgaatcataatctgtattttawccagatctccaggcataggtattatacattaaaggtagagacacacatctctaagagatttgttacctgataggtactttattctctaatcctaggtaagggctagaaagcagtgttctatcattgtcagccgggaaatctttcccccttctctccactgaaccacacttttaacttgtctctgttcagaaattttccac

SEQ ID NO: 39 - DNA, 801 bp, Homo sapiens
tctggaagactggctgtgaaaagtcaggtggcagaaacctggggccacatagagcctctctcttttcctgtttcttggctctagaagatcagcactgcactgttagctgagagtgcgggcaagacataaactgtccagagtttgaaggttctcggaaagaccggagggcttctccccacagaaggcggagagagctggggctcagacatgggtgtgcaccttaataaaccctgctgtctgcctccctgactctgcttcttgggagcatggtgagcagccctggtgctcagcagccatacctatgggacacacactacgaaaaggatgcctttagggtttgggggagattttactcctttcttcaacaactattcactggacaagttctctgctcccatgaygcgccaggcacagttctgcaagtatattgtgaatgtattgttctagtgggatacacaaataagtcagttaaaatacataaataaaaacataaacctgccatagattcgacatttgctggtgatggacgtcggcaggatgatttgaagaaactaactagatgaggggagagggcagtgaaggagactttagacaggatggttgggaggtgagatctgaactgaaacccagaggaggagagtctgaggactagtgttcccagaaggcagatgccgatgactgacctgagggccttcctccaagacttcattgtcatatgatgtcattagagaggtgtcaatgtggagagaagctgaccacataagggccttgaaatgtgccatcctggagaggccaccccaa

SEQ ID NO: 40 - DNA, 654 bp, Homo sapiens
atgttttgagtggttttagcttgattctaccaaatgacatcatggagcaagagcactgcgaagggttatagcaaagggaaatagggtataagaaggctttcctatgcgcttgacttgcttttgagttagggaccctctcagccactgttactactctttcttttaagtagattcctacatttgtaattgtggagaagcagtagcacaagtgattgagagcrcaggctctgggatcaagcagcttagggttcagattatgctccacaagctgtgccagcgatatggcatcagacaaagtactaacctccctcttccagttttctttccaaaatggggtaatgacagtccctccctcgtaaggttgttgtgagaattaaatgagatgagttgattaatgcaaagatttgcacagtgtctggaaacgttgtttgcacttggtacctgtgagttattacctgagcctgtggcccttgatggaaaaagcttataagcccccaccagacaggactggaaacagaatgatacatgtttccttttggttggcctgccaacccccttcacacacacacacaaagacacactctgtcctgcaatggaagggcccattgctaaatgatgatgcctgctggaaggatcccaggcagtggtgacaca

SEQ ID NO: 41 - DNA, 1314 bp, Homo sapiens; identical to SEQ ID NO: 38

SEQ ID NO: 42 - DNA, 801 bp, Homo sapiens; identical to SEQ ID NO: 39

SEQ ID NO: 43 - DNA, 801 bp, Homo sapiens
tttttttttaaccacttttaatgtggctactaacacatctaaaatttcacatgtggttcacattatgtttatattggacagtgcagtcctagactccctgttttagagttaaggaaaccaagttcaaagccttgtccgaaatcaagtttgaggttgtttgaatccagatctatctgatatcaagtgtgggcacttcactgctgctctctctagaatctaaactttccgatgggcagaggtaagagctaaaaggatcgcccttgggaagaaagagaggaaggagaaaagaagtgagggaggaaatggcactcatgagaatgttctctctgaccactcctcttgcccttcaggaggccctgtccccaggggagccactggttgtgtccaccggagatctgcarctcctgtacttctatgctgggcaatgccagagccactactcagccctgcaggcagccgtggcagccctgatgtccagtacccaggctaatcagcccccgcgccttttcgtgccccacagcaagagggtggtggtggctgctcatcgcctggtgtttgttggggacaccctgggccggctggcagcctctgcccctctgagagcacaggtcagggctgcaggtacagcactgggccaggcattgcgggccactgtgctggctgtcaagggagctgccctgggctacccatccagccctgccatccaagagatggtgcagtgtgtaacagaactggcagggcaggccctgcaattcactaccctgctcactagcctggctccatgaaggtcctttggcacag

SEQ ID NO: 44 - DNA, 1001 bp, Homo sapiens
ttagattctagagagagcagcagtgaagtgcccacacttgatatcagatagatctggattcaaacaacctcaaacttgatttcggacaaggctttgaacttggtttccttaactctaaaacagggagtctaggactgcactgtccaatataaacataatgtgaaccacatgtgaaattttagatgtgttagtagccacattaaaagtggttaaaaaaaaaacaaaaccctaatatattttatctaactccaaatataaaaatgttataatttcaacatataatcaacatgaaaattattgagatttttcccatactgagtctgtaaaatccaggctgcattttatatttgcagcatatttcaatttgaactaggcctatcttgagtgctcaatagccatatgtagctagtggctactgtactagatagtgcagttctagtatgtacctcatgaggctgcagtgaaggtcaattgagataatatacagaaagggcttagcamagtgcatttcacactgtgagcaccagatgagtgttaactgtggctgtgattaatactaacacgtgcagtgtgcctagcacagggtctggcacttggtaaatacctgataaatggtagctattgtgttgcgaccattctacgcagggttgaggaaaccacctcactttctgaggcctggagagattaactgacggcttcaaagcccctggcttctcagtagtgaagctgggattagaagccaggtctcttaggagcccaggaattcacagttggaaggaggctcagaagtccccaggcccagctctgaccccacaactcagaaactttctgctctcctggctgcttgattttctgactcctctccaacacattctttcccctctttctgaattcctaacatcctcttccaaggtccagttccttcctgtagatttctaacttgcctccgctctgtccctggttcctgtgaactctcactccatcctaacctatttccttcc

SEQ ID NO: 45 - DNA, 801 bp, Homo sapiens
ctcctcgcactcagacattatctgatcaccggcccctccgttcctcctgaccccccatctatttgatccctcaggtgtatgtggtgccgcccccagctcggccctgtccaacctcaggacctccagctggaccttgcccaccctctcctgacctcatctacaaaatccccagagctagtgggacccagctggctgctcccagagatgccttggaggtgagggctagaggtccctgtgtatgttgggtgggagacaaatggccccaagggagggttcatcgctgagctgacagatgactgaccactcctttccgcaggtctacgatgtgccccccaccgccctccgggtgccctccagtggcccctatgactgccctgcctccttttcccaccctctgaccygggttgccccgcagccccctggagaggatgatgctccctatgatgtgcctctgaccccaaagccacctgcagagctggaaccagatctggagtgggaaggaggccgggagccggggccccccatctatgctgccccctccaacctgaaacgagcgtcagccttactcaatttgtatgaagcacccgaggaactgctggcagacggggagggcgggggcactgatgaggggatctacgatgtgcctctgctggggccagaggctcccccttctccagagccccctggagccttggcctcccatgaccaggacaccctggcccagcttctggccagaagccccccacccccacacaggccccggctcccctcagctgagagcctgtcccgccgccctctgcc

SEQ ID NO: 46 - DNA, 201 bp, Homo sapiens
acagggagagttagaaggtctctaagctcctcgcactcagacattatctgatcaccggcccctccgttcctcctgaccccccatctatttgatccctcagrtgtatgtggtgccgcccccagctcggccctgtccaacctcaggacctccagctggaccttgcccaccctctcctgacctcatctacaaaatccccagagc

SEQ ID NO: 47 - DNA, 2464 bp, Homo sapiens
tgggcttgggtgctgggccagcaggcaagagcttcaccctgttggcgggcacaatgccctgctggccgtgtagggagcagaggcaccagccgtccagtccaccagcgccctctctctgcaggacccgtaggacatcccctcggcggaaggacagctcctggggggactcagcggtgttgtcatacagtgcccgggccagctgggtctggttggggaggtgggagtggggagaagggtcttcagacccctttcaggtcacgtcacccctgcttagaacccttcggagctccgcatttccctcaagctaaaaaacctgaactctggatgcagctttctgcctgggccctagcttctctccagcttcaccgcttgcattctccctcttcctttcagttccttcaactagaccagctctcctcagagaggccgattctttactcttctccccgccccattctctgtttgtctccacctattgtttccatcctcgtactttatactatttgtctatttgcttcttttgtatcgatttcttcccttatactgtaaaccctatgaacataggaacggctatatttctagtccttgcaataaggcctgacctggaattggtcattacacatgttgaatgaatgtcttccaaaacatrtgaagccctgctacagtctctcatagaccttctctctgttcccaagtcattatccctcactagcaaaaggttagattaagcagccacagagacctctcattctgagagactgggagaggcctagtccagacagtgtgcctaggcataataagaaagaaaacagaaacaaagcaattgttttgctctcagtctgagcaaataagtttctaccagaagaggctgaggttaccatggagaaaagtgtgtattcttgtgtgtgtgtcaccactgcctgggatccttccagcaggcatcatcatttagcaatgggcccttccattgcaggacagagtgtgtctttgtgtgtgtgtgtgaagggggttggcaggccaaccaaaaggaaacatgtatcattctgtttccagtcctgtctggtgggggcttataagctttttccatcaagggccacaggctcaggtaataactcacaggtaccaagtgcaaacaacgtttccagacactgtgcaaatctttgcattaatcaactcatctcatttaattctcacaacaaccttacgagggagggactgtcattaccccattttggaaagaaaactggaagagggaggttagtactttgtctgatgccatatcgctggcacagcttgtggagcataatctgaaccctaagctgcttgatcccagagcctgcgctctcaatcacttgtgctactgcttctccacaattacaaatgtaggaatctacttaaaagaaagagtagtaacagtggctgagagggtccctaactcaaaagcaagtcaagcgcataggaaagccttcttataccctatttccctttgctataacccttcgcagtgctcttgctccatgatgtcatttggtagaatcaagctaaaaccactcaaaacatacaattacattcagcttcagttttgattgctaaatgatgatgcttgctggtaggatcccaggcagtggtgacttacatgtagtgttggagagcagctagttaggcattactagccaggtagccaaacggactgactaattcagcaaaaaaatctcaaagttaaagatctcaggcaggctgccaccagaccctggggagaaggatgccctgctagagtcagcaggagtgcatattcctcttagctgatgtttccctctggtggtgaggttatccggcctctcatggctgggtgattttaacaacaaattgacccctcttcttgcctgattcccaactcatcaaaccattttctgattatattttggtttctaaaggatattgtcccacacaaagaaacgtgctaaccagatgacacaaattcccggttagtgctgcacttttctttttagttgtaaattataaatcagtcttttggatctagccaggggattaaaagcacagttctagaggcagacctgggcttgagtcttggctgtgtctctttcttttaaaattttatttaaaatatttctttagagatcagctcttgatctgtggtccagactggagtgcagtggcacaatcataactcactgtaacctcgaatgcctgggttcagcctcctaaatacctgggattaccgttgcgacccactgcttggaggtctgtccctttctttttctttctttcttttttttttttttgagacggagttttgctctgtcgcccaggctggagtgcagtggtgcgatctcggctcactgcaagctccgcctcccgggttcacgccattctcctgcctcagcctcccgagtagctgggactacaggcgcccaccaccacgcccggctaattttttgtattt

SEQ ID NO: 48 - DNA, 654 bp, Homo sapiens; identical to SEQ ID NO: 40

SEQ ID NO: 49 - DNA, 480 bp, Homo sapiens
aactggatcatggttttctgacttagtgatatggagaccactggttgtttcagaacaaatggaataagcattaggcaggagtttaaactttgcttatccaccatgagaaaatacaatgcctaattttggggggatatttataaagggcacggaaatagctacaaatgagactattcatttgaacatgtctgtttcaaatgctaacayattgagcgaatttgatagtcccccagttttgctggtcctcagtttcttcaaaataagatgaaaggattgaactgtaaaagtaggaaagttctaactcaccatcccctgaccacacaaacacacacaacttcaccttccttaatgtgtgactgaaactacgaaaccacttctagcctgcaatttctattcctatcacaatatattacctttctatttagccatagggctatcacttgatgttagtggcacaattagacatcacttcaagttctg

SEQ ID NO: 50 - DNA, 601 bp, Homo sapiens
agctgatcagaaaggagtgccagccacaaacaactgtatagtctcgtaactcccagctgagcaagtgtagttgctgagctgaagagagtaggtaatttttgaaaccaatcagccatttaaaaaaaaagagggttatgattatgaatattttataaaatatcattatttcttatttttaaataccatatgttttgattataaccattatcatgaggaaaatctcacctgagtttcactatgtctccattgaaaaattcttacaaatttatggaaacttcaaagtggttgcaaccattcaaakaaatgaatgactgtaatgaatgcagattgccttaacttgagctaagaaagtgtcagagtttaacatagaaattaaacaaatgattttttgagaaactaaaatgattactaacggtataataaaccaataattttctgtgacatcaaccaggattttagcttctatcagaaaatactatcacagacaaacaacaagcaaaccaaaacttcagacgcatgctggggaggctacagaaggaatgaagtctttacactcaggagcaaaaaggcatccagatggtatgttctgccttactttaga

SEQ ID NO: 51 - DNA, 601 bp, Homo sapiens
atagaaattaaacaaatgattttttgagaaactaaaatgattactaacggtataataaaccaataattttctgtgacatcaaccaggattttagcttctatcagaaaatactatcacagacaaacaacaagcaaaccaaaacttcagacgcatgctggggaggctacagaaggaatgaagtctttacactcaggagcaaaaaggcatccagatggtatgttctgccttactttagaaggctggaatggtagagaggaggggagagatgatggccaccagggaagggaaataatctgtctckcaaatcttcccttgaaattagagtttagggatcccataaggcagtggtttgtacatagtcagggcaaatccacacacagcaatagcagccatggaaccatacttcaaggatcttgggaggacattgacttgcataccttgggaaatcacacggacgacatcagaaaacccaggtagcatactttcccatcataaactagctcacatacactcacaaaatcagaaatgtggtgacatgaggttagggtgggggagttccacctcatatagaaagtattcatttaatgtattatgtaatgtc

SEQ ID NO: 52 - DNA, 601 bp, Homo sapiens
aggatgtttatttgattagtaacagaaattctaaaaggctatgtgtaccctctctgtattatactatattataagaccaacactaaaggtggatgtgaggaagtcgtaatactgattgaacgagttctttaaaatcagcaatagcaactcattcatcattcatctccaaccatagcacattgtctatttgactcaataaatacatgataactgttttttaattttaatttacccttggcaactcaactgggagctctgaattaagtattgcccatcagtttcccatgtcaggtcaaaatgkccagcaatctatacactttccttgctcttaaaactttccttgaacttaaaactattgaagtttggccttggaataaaatcatcagtctctgaggaaaaaacttatataatcgacttctgtcaatgatcactaatgccacaaatctagatctctcatttggagtctcaaatggcagatgaatttgattagctcctatgactataagctaagctaattattacaagtggatgtggagaaaacagtcctatggtggcctgtgctatctgatcttgaacttttccatttcttcctttactgttt

SEQ ID NO: 53 - DNA, 601 bp, Homo sapiens
gaggtagggattaaatgccttatccaccaatggtaacccacagctagagaacaaaccaattaattgcttccttctctttcctgtctaactctttcacccctctactatgttaggaacacctccaaataaactacttgcactcaaatcgttgaactgggggttgcttctgagggaacccagtctaaaacggaactagagatgagtgtggtgagagactgatgtaggtacatgctcaatttcttcaagtttggattgaagaagagttttatatttatcataagcttttctagactcaagatgyaataacacaggggcaggtgggaagtgggatggaagactgaaactcaggtggtcataaaacatagtctctatctttatttcaatgctttttaaaataagtgattgtgtgtgtgtgtgtgtgtgtgtgtgtgtgtgtgacagagggagaggtggcagcggggagagcataagactgtggtgttcagatttctgttttgtgagtctccagaaatgaatcttagggccttcactgccatcaaggactatcaatcaatcaggggatagactttgaggtagatacagtaggtctgctcactttaca

SEQ ID NO: 54 - DNA, 594 bp, Homo sapiens
tcacatggctgcataatctcagccactctttcacaactatagactcatacacgcgaagtgccagattcatgcacaaccacacaatcacatggaagtcacagacggcatcacagacagtcacagcactgtgtgtatgttataacacaagcacacaaaactcagacagcatcccagctacacagccactcccagaggtgtcascgtcacacttggtaattaatactcattacattagacacagacagaccaagttatagtcagacctggttacacacatacacacacacaatatcaccatgacaaatacacattacacacacacaacatcacaatgacaaacacacattacacacacaacatcacgatgacaaacacacattacacacacaacatcacgatgacaaacacacattacacacacatcacaatgacaaacacaacattacacacacacaacatcacaatgacacacacatcacacacacatcacaatgacaaacacacaacattacacacatatacacacagcctgagggccctccccagcccagactaacacatctcggggtgaggaccagaccttgttcataaccc

SEQ ID NO: 55 - DNA, 781 bp, Homo sapiens
gctggggcggtgtgggctgcccgctggggcctgggccagcaccatcacccacgggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggagraggtggtcgtccgtgtgctggatgaacgcctggttcgactgcgtgatggtacccggtcttacttcggggctttcatggtgtgaaggaaggagcgtggtgcattggacatgggtctgacacgtggagaactcagagggtgcctcaggggaaagaaaactcacgaagcagaggctgggcgtggtggctctcgcctgtaatcccagcactttgggaggccaaggcaggcggatcacctgaggtcaggagttcgagaccagcctggctaacatggcaaaaccccatctctactaaaaatacaaaaattagccggacgtggtggtgcctgcctgtaatccagctactcaggaggctgaggcaggataattttgcttaaacccgggaggcggaggttgcagtgagccgagatcacaccactgcactccaacctgggaaacgcagtgagactgtgcctcaaaaaaaagaaaggaagaaaaaagaaaactcaggaaacagatcttgggggacactccagggaacccaaaactcaaaggcggagagctcagtgggcaccaccaaggcgagatgaagccccagcaggcac

SEQ ID NO: 56 - DNA, 825 bp, Homo sapiens
ctggctgtggatgtgtccgcctgtctgtgtgcctcacctgggggtctatgtgtgcccaacaccatacccctgtgggcttggccccttttgtgtcctgggtctggatccggtggccacacattcgtgtccatatacccatgtggacgtatgtgcrtgtacccacatgtccacacacccgcctagctgcagccggtgtattcacgttgtctgtctttgtctctgggtctctgtgtctgtagacgcacatggatccataaaccacaaaccacatgtccacgcatctgagcccacatgtgtcttctcagcgtcatgatgatgttgaggtctgtgccctgtggcaggtgctatatcccaagggtggtgtccatgtgtccagccatctcgctgtctgcatttttttttttttgagacaaggtttctctcccatagcccaggctgtagtgtagtggtgcagtcttggctcactacaaccccacctcctaggttcaagtgattcccttgtctcagcctcctgagcagtagctgggatcacaggcattcgccaccgtgcctggctaatttgtgtatttttagtacagacggggtttcaccatgttggccagacttgtctcgaactcctgacctcaagtgatccacctgcctcagcctcccaaagtgctgggattacaggtgtgagccacacgcctgggcttgcccctgttttttttaatttacttattgatttgaacaatacatatttatttatcttaaaaaactggggtttcactctgcttcccagctagaatgggggggaacgacatagtttattgaagcttgaactcttgg

SEQ ID NO: 57 - DNA, 2478 bp, Homo sapiens
ggggttcagccccaccccttcccccactcaccctcctcttcttccggtacccgccgcccccggtcttggccctgtctcactctcactcatacagactctcacactttccagaggcttctgaaaatgtgactcaggtggcaagtgcagtggggagcccccagctttcccttcttggatgcttcattcgcttggggccaccaaatatcgactgaggactttctgcccatgccaggctctgctctcggtgtgggggatgcagcaatgaacaacagcaagaagggtccctgctcctcttctggtggagccagagagacaagaaacctcgtaaacaagaaaataatacgttgtgggtttttttgttcgtttatttgttttgtttttgagatggagtctcgctctgttgcccaggctggagtgcagtggcgcgatctcagcttactacaacctccacctcctgggttcaagcaattctcctgcctcagcctcccaagtagcggggattacaggcgcgtgccaccacgcctggctgctttttgtatttttagtagagatgggttttcaccatgtcggtcaggctggtctcgaactcctgacctcaagcgatccacccacctcagcctcccaaagtgctgggattacaaacgtgagccgcaccatgcccggccttctgttgtgttttgatgtgacaagtgcagtgaaaaacacaagagtggaataaaggagtggatgtgtgctctgtgtgtgtgcttgtctgcatgcatatatgtgtgcagtgtgtgcatgtgaatgtgtgtattcagttgtgtgcatgcaagtgtgtatcatgtacacatgtgcaggcgcatgcctgtgtatcatacatgtgctggcatgtaaatgtgcgtgcatatgtgggcaggtgcatgcatatgtgtatggtcatctgtgtgcatgtgcaygtggtccatgtacatgcatgcatgtgcacgtgtgtgtatgtgtgaacatgcatcacatgtgcaagtgggtgtgcatatatgtgctgtgtgcaggtgtgtgcatgtgtgtgtgcctgtgcattggtgcgtgtatgtgtgtgtgcgcacttgcacgggtgtgttcatgtgtgtggggaggtccatgtatgagtgtgcatgcattcaagcgtggatttttttttttttcgagacggagtctcgctctgtcgcccaggctggagtgcagtggtgcgatctcggctcactgcaagctccgcctcccaggttcacgccattctcctgcctcagcctcctgagtagctgggactacaggcgcccaccaccgcacccagctaattttttgtatttttagtagagacggggtttcaccgtgttagccaggatggtctcaatctcctgacctcgtgatccgcctgccttggcctcccaaagtgctgggattacaggcataagccaccgcacccagcctttttctttctttttttttttgaggcagagtctcactttgtcacccaggctggagtgcagtggtgccattttggctcactgcaacctctgcctcccggattcaagcaattttcctgcctcagcctcctgagtagctgggattacaggcgcccaccaccatgcatagctaatttttgtattatatgttggccattatatatgttgtaacatatatgttgtctatatgttggccaggctggtctcgaactcctgacctcaggtgatctgcccacctcagcctcccaaaatgctagaattacaggcgtgagccactgcacttggcttctgtcttgtttcataagtatctttgtaatatccttgattttacctcttggccctgaaagccaaaaacacttatgtggccctttatagatacagtttgctgccagcttgcccctgagcttaggctaagacacgaagtgcccttcactggggcctctgtggttggaccctcgtgcttttgctcactctgccccagacagatatcctgggtctttgcacttcctcttcctgctgctgggaaccatcttccctagatctccaaagcaggctccccctgctcatctgcgtcttgactcaagaaacttctcacaggagccctccctgaattggtcaggtgtctcccgagagacagaaccaatagaagatacacagagagatagagatgggatctataggctgggtgcggtggctcacgcctgtaatcccagcactttgggaggctgaggcatgtggatcacctgaggtcgcgagttcgaggccagcctggccaatatggggaaaccctgtctctatcaaaaatacaaaaataaaaaataaaaaattagctaggtgtggtggcacacttctgtaatcccagctacttgggaggctgaggctcaagaatcacttgaacccgggaggcacaagttgcaatgaactgagattgtgccactgcactccaggctgggcaatggcatgagactgtctcaaaaaaaaaaaaaaaaaaagagat

(19) United States
(12) Patent Application Publication
(10) Pub. No.: US 2010/0240043 A1
(43) Pub. Date: Sep. 23, 2010

(54) METHODS OF USING GENETIC VARIANTS TO DIAGNOSE AND PREDICT INFLAMMATORY BOWEL DISEASE

(75) Inventors: Jerome I. Rotter, Los Angeles, CA (US); Kent D. Taylor, Ventura, CA (US); Stephan R. Targan, Santa Monica, CA (US); Dermot P. McGovern, Los Angeles, CA (US); Ling Mei, Pasadena, CA (US); Xiaowen Su, Beverly Hills, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/675,718

(22) PCT Filed: Oct. 20, 2008
(86) PCT No.: PCT/US2008/080526

§ 371 Date: Feb. 26, 2010

Related U.S. Application Data
(60) Provisional application No. 60/981,356, filed on Oct. 19, 2007.
(60) Provisional application No. 60/981,385, filed on Oct. 19, 2007.
(60) Provisional application No. 61/054,578, filed on May 20, 2008.
(60) Provisional application No. 61/055,262, filed on May 22, 2008.

(51) Int. Cl. C12Q 1/68

(52) U.S. Cl. 435/6

(57) ABSTRACT

The present invention relates to methods of diagnosing susceptibility to Inflammatory Bowel Disease and subtypes of Inflammatory Bowel Disease. In one embodiment, the present invention provides a method of diagnosing susceptibility to Inflammatory Bowel Disease by determining the presence of one or more risk variants at the DR3 locus, GATA3 locus, SIN(EFS) locus, BTLA locus, LIGHT locus and MAGE locus.

### FIELD OF THE INVENTION

The invention relates generally to the fields of inflammation and autoimmunity and autoimmune disease and, more specifically, to genetic methods for diagnosing inflammatory bowel disease, Crohn's Disease, ulcerative colitis and other autoimmune diseases.

### BACKGROUND

All publications herein are incorporated by reference to the same extent as if each individual publication or patent application was specifically and individually indicated to be incorporated by reference. The following description includes information that may be useful in understanding the present invention. It is not an-admission that any of the information provided herein is prior art or relevant to the presently claimed invention, or that any publication specifically or implicitly referenced is prior art.

Crohn's-disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise, etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K: Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. R Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36,471 (2004)). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

### SUMMARY OF THE INVENTION

Various embodiments include a method for diagnosing susceptibility to Inflammatory Bowel Disease in an individual, comprising determining the presence or absence of a risk haplotype at the DR3 locus in the individual, and diagnosing susceptibility to Inflammatory Bowel Disease in the individual based upon the presence of the risk haplotype at the DR3 locus. In another embodiment, the risk haplotype at the DR3 locus comprises DR3 H2. In another embodiment, the individual is non-Jewish. In another embodiment, the risk haplotype at the DR3 locus comprises SEQ. ID. NO.: 7, SEQ. ID. NO.: 8, SEQ. ID. NO.: 9, SEQ. ID. NO.: 10, SEQ. ID. NO.: 11, SEQ. ID. NO.: 12 and/or SEQ. ID. NO.: 13. In another embodiment, the Inflammatory Bowel Disease, comprises Crohn's Disease and/or ulcerative colitis.

Other embodiments include a method of determining a low probability of developing Inflammatory Bowel Disease in an individual, relative to a healthy individual, comprising determining the presence or absence of DR3 H1 in the individual, and diagnosing a low probability of developing inflammatory Bowel Disease in the individual, relative to a healthy subject, based upon, the presence of DR3 H1. In another embodiment, the individual is non-Jewish. In another embodiment, the DR3 H1 comprises SEQ. ID. NO.: 7, SEQ. ID. NO.: 8, SEQ. ID. NO.: 9, SEQ. ID. NO.: 10, SEQ. ID. NO.: 11, SEQ. ID. NO.: 12 and/or SEQ. ID. NO.: 13.

Other embodiments include a method of determining a low probability of developing Crohn's Disease in an individual, relative to a healthy individual comprising determining, the presence or absence of a protective haplotype at the DR3 locus in the individual determining the presence or absence of a protective haplotype at the TL1 A locus in the individual, and diagnosing a low probability of developing Crohn's Disease in the individual, relative to a healthy subject, based upon the presence of the protective haplotype at the DR3 locus and the presence of the protective haplotype at the DR3 locus. In another embodiment, the protective haplotype at the DR3 locus comprises DR3 H1. In another embodiment, the protective haplotype at the TL1A locus comprises TL1A H2. In another embodiment, the protective haplotype at the DR3 locus comprises SEQ. ID. NO.: 14, SEQ. ID. NO.: 15, SEQ. ID. NO.: 16, SEQ. ID. NO.: 17, SEQ. ID. NO.: 18 and/or SEQ. ID. NO.: 19. In another embodiment, the individual is non-Jewish.

Various embodiments include a method of diagnosing susceptibility to Inflammatory Bowel Disease in an individual, comprising determining the presence or absence of a risk haplotype at the GATA3 locus in the individual, and diagnosing susceptibility to Inflammatory Bowel Disease in the individual based upon the presence of the risk haplotype at the GATA3 locus. In another embodiment, the risk haplotype at the GATA3 locus comprises GATA3 Block 2 Haplotype 1. In another embodiment, the Inflammatory Bowel Disease comprises Crohn's Disease and/or ulcerative colitis. In another embodiment, the risk haplotype at the GATA3 locus comprises SEQ. ID. NO.: 22, SEQ. ID. NO.: 23, SEQ. ID. NO.: 24, SEQ. ID. NO.: 25, SEQ. ID. NO.: 26, SEQ. ID. NO.: 27 and/or SEQ. ID. NO.: 28.

Various embodiments include a method of diagnosing Crohn's Disease in an individual, comprising determining the presence or absence of a risk haplotype at the GATA3 locus in the individual, determining the presence or absence of Th1/Th2 dysregulation, and diagnosing susceptibility to Crohn's Disease in the Individual-based upon the presence of the risk haplotype at the GATA3 locus and the presence of Th1/Th2 dysregulation. In another embodiment, the risk haplotype at the GATA3 locus comprises GATA3 Block 2. Haplotype 1. In another embodiment, the individual is non-Jewish. In another embodiment, the risk haplotype at the GATA3 locus comprises SEQ. ID. NO.: 22, SEQ. ID. NO.: 23, SEQ. ID. NO.: 24, SEQ. ID. NO.; 25, SEQ. ID. NO.: 26, SEQ. ID. NO.: 27 and/or SEQ. ID. NO.: 28.

Other embodiments include a method of diagnosing susceptibility to Crohn's Disease, comprising determining the presence or absence of one or more risk haplotypes at the TL1A locus, TLR5 locus and NOD2 locus, determining the presence or absence of a high expression relative to a healthy subject of anti-OmpC expression, and diagnosing susceptibility to Crohn's Disease in the individual based upon the presence of one or more risk haplotypes at the TL1A locus, TLR5 locus and NOD2 locus and the presence of high expression relative to a healthy subject of anti-OmpC expression. In another embodiment, one of the one or more risk haplotypes comprises TL1A Haplotype B. In another embodiment, one of the one or more risk haplotypes composes TLR5 Haplotype 2. In another embodiment, the individual is Jewish. In another embodiment, one of the one or more risk haplotypes comprises SEQ. ID. NO.: 29, SEQ. ID. NO.: 30, SEQ. ID. NO.: 31, SEQ. ID. NO.: 32 and/or SEQ. ID. NO.: 33. In another embodiment, one of the one more risk haplotypes comprises SEQ. ID. NO.: 34, SEQ. ID. NO.: 35, SEQ. ID. NO.: 36 and/or SEQ. ID. NO.: 37.

Various embodiments include a method of diagnosing susceptibility to a subtype of Crohn's Disease in an individual, comprising determining the presence or absence of at least one. risk haplotype in the individual, selected from the group consisting of TL1A Haplotype B and TLR5 Haplotype 2, and determining the presence or absence of a high expression relative to a healthy subject of anti-OmpC expression in the individual, where the presence of one or more risk haplotypes and the presence of high expression relative to a healthy subject of anti-OmpC is diagnostic of susceptibility to the subtype of Crohn's Disease in the individual. In another embodiment, the presence of two of said risk haplotypes presents a greater susceptibility than the presence of one or none of said risk haplotypes, and the presence of one of said risk haplotypes presents a greater susceptibility than the presence of none of said risk haplotypes but less than the presence of two of said risk haplotypes. In another embodiment, the individual is Jewish.

Other embodiments include a method of diagnosing susceptibility to a subtype of Crohn's Disease in an individual, comprising determining the presence or absence of one or more risk haplotypes at the SIN(EFS) locus, determining the presence or absence of a high expression relative to a healthy subject of anti-Cbir1 expression, and diagnosing susceptibility to the subtype of Crohn's Disease in the individual based upon the presence of one or more risk haplotypes at the SIN(EFS) locus and the presence of high expression relative to a healthy subject of anti-Cbir1 expression. In another embodiment, one of the one or more risk haplotypes at the SIN(EFS) locus comprises SIN(EFS) haplotype 2, In another embodiment, one of the one or more risk haplotypes at the SIN(EFS) locus comprises SEQ. ID. NO.: 38, SEQ. ID. NO.: 39, SEQ. ID. NO.: 40, SEQ. ID. NO.: 41, SEQ. ID. NO.: 42, SEQ. ID. NO.: 43, SEQ. ID. NO.: 44. SEQ. ID. NO.: 45, SEQ. ID. NO.: 46, SEQ. ID. NO.: 47 and/or SEQ. ID. NO.; 48.

Various embodiments include a method of diagnosing susceptibility to a subtype of Crohn's Disease in an individual, comprising determining the presence or absence of one or more risk haplotypes at the BTLA locus, determining the presence or absence of a high expression relative to a healthy subject of anti-I2 expression, and diagnosing susceptibility to the subtype of Crohn's Disease in the individual based, upon the presence of one or more risk haplotypes at the BTLA locus and the presence of high expression relative to a healthy subject of anti-I2 expression. In another embodiment, one of the one or more risk haplotypes at the BTLA locus comprises BTLA Block 1 Haplotype 1. In another embodiment, one of the one or more risk haplotypes at the BTLA locus comprises SEQ. ID. NO.: 49, SEQ. ID. NO.: 50, SEQ. ID. NO.: 51, SEQ. ID. NO.: 52 and/or SEQ. ID. NO.: 53.

Other embodiments include a method of diagnosing susceptibility to a subtype of Crohn's Disease in an individual, comprising determining the presence or absence of one or more risk haplotypes at the LIGHT locus, determining the presence or absence of a high expression relative to a healthy subject of anti-I2 expression, and diagnosing susceptibility to the subtype of Crohn's Disease in the individual based upon the presence of one or more risk haplotypes at the LIGHT locus and the presence of high expression relative to a healthy subject of anti-I2 expression. In another embodiment, one of the one or more risk haplotypes at the LIGHT locus comprises LIGHT Block 2 Haplotype 2. In another-embodiment, one of the one or more risk haplotypes at the LIGHT locus comprises SEQ. ID. NO.: 54, SEQ. ID. NO.: 55, SEQ. ID. NO.: 56 and/or SEQ. ID. NO.: 57.

Various embodiments include a method of determining a low probability of developing a subtype of Crohn's Disease in an individual, comprising determining the presence or absence of one or more protective haplotypes at the BTLA locus, and diagnosing a low probability of developing the subtype of Crohn's Disease in the individual, relative to a healthy individual, based upon the presence of one or more protective haplotypes at the BTLA locus. In another embodiment, one of the one or more protective haplotypes at the BTLA locus comprises BTLA Block 1 Haplotype 3. In another embodiment, the subtype of Crohn's Disease comprises a small bowel surgery phenotype. In another embodiment, one of the one or more protective haplotypes at the BTLA locus comprises SEQ. ID. NO.: 49, SEQ. ID. NO.: 50. SEQ. ID. NO.: 51, SEQ. ID. NO.: 52 and/or SEQ. ID. NO.: 53.

Various embodiments include a method, of determining a low probability of developing a subtype of Crohn's Disease in an individual, comprising determining the presence or absence of one or more protective haplotypes at the LIGHT locus, and diagnosing a low probability of developing the subtype of Crohn's Disease in the individual, relative to a healthy individual, based upon the presence of one or more protective haplotypes at the LIGHT locus. In another embodiment, one of the one or more protective haplotypes at the LIGHT locus comprises LIGHT Block 1 Haplotype 3. In another embodiment, the subtype of Crohn's Disease comprises a fibrostenotic phenotype. In another embodiment, one of the one or more protective haplotypes at the LIGHT locus comprises SEQ. ID. NO.: 54, SEQ. ID. NO.: 55, SEQ. ID. NO.: 56 and/or SEQ. ID. NO.: 57.

Other embodiments include a method of diagnosing susceptibility to a subtype of Inflammatory Bowel Disease in an individual, comprising determining the presence or absence of one or more risk haplotypes at the MAGI2 locus in the individual, and diagnosing susceptibility to the subtype of Inflammatory Bowel Disease based upon the presence of one or more risk haplotypes at the MAGI2 locus in the individual. In another embodiment, one of the one or more risk haplotypes at the MAGI2 locus comprises a variant listed in Table 3, Table 4 and/or Table 5 herein.

Various embodiments include a method of determining a low probability of developing a subtype of Inflammatory Bowel Disease in an individual comprising determining the presence or absence of one or more protective, haplotypes at the MAGI2 locus, and diagnosing a low probability of developing the subtype of Crohn's Disease in the individual, relative to a healthy individual, based upon the presence of one or more protective haplotypes at the MAGI2 locus. In another embodiment, one of the one or more protective haplotypes at the MAGI2 locus comprises a variant listed in Table 3, Table 4 and/or Table 5 herein.

Other embodiments include a method of diagnosing susceptibility to a subtype of Inflammatory Bowel Disease in an individual, comprising determining the presence of one or more risk variants at the DR3 locus, GATA3 locus, SIN(EFS) locus, BTLA locus, LIGHT locus and MAGI2 locus in the individual, and diagnosing susceptibility to the subtype of inflammatory Bowel Disease in the individual based upon the presence of one or more risk variants at the DR3 locus, GATA3 locus, SIN(EFS) locus, BTLA locus, LIGHT locus and MAGI2 locus.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts haplotype nomenclature of DR3, with “1” designating the major allele and “2” designating the minor allele. The haplotype block was constructed by Haploview.

FIG. 2 depicts haplotype nomenclature of TL1A, with “1” designating the major allele and “2” designating the minor allele. The haplotype block was constructed by Haploview.

FIG. 3 depicts a graph of the haplotype of DR3 and CD in non-Jews.

FIG. 4 depicts the cumulative effect of protective haplotypes of DR3 and TL1A in non-Jews.

FIG. 5 depicts GATA3 haplotype block and haplotype structure. The “1” denotes the major allele and the “2” denotes the minor allele.

FIG. 6 (a) and (b) depict charts of the association between GATA3 haplotype carriers and IBD, with (a) depicting Crohn's Disease and (b) depicting ulcerative colitis.

FIG. 7 (a)-(c) depict charts of the association between GATA3 haplotype and IBD in non-Jews, with (a) depicting Crohn's Disease, (b) depicting ulcerative colitis, and (c) depicting IBD.

FIG. 8 depicts qualitative results, demonstrating TL1A HB and TLR5 H2 was positively associated with anti-OmpC expression in. Jews.

FIG. 9 depicts quantitative results, demonstrating TL1A HB and TLR5 H2 was positively associated with anti-OmpC expression in Jews,

FIG. 10 depicts haplotype structures of TL1A and TLR5 genes. The “1” represents the major allele, and “2” represents the minor allele.

FIG. 11 depicts qualitative results, demonstrating NOD2 by itself was not associated with anti-OmpC in Jews, but risk haplotypes or variants from the three genes combined to increased anti-OmpC expression.

FIG. 12 depicts quantitative results, demonstrating NOD2 by itself was not associated with anti-OmpC in Jews, but risk haplotypes or variants from the three genes combined to increased anti-OmpC expression.

FIG. 13 depicts a chart describing results where Jews, in sum by quartile of the expression of all antibodies also increased with increasing number of genetic risk factors.

FIG. 14 depicts a chart depicting three SNPs found to be associated with the presence of anti-Cbir1 antibody expression in CD subjects: rs7148564, rs2231810 and rs2231805 (R175W).

FIG. 15 depicts a haplotype block observed for SIN(EFS).

FIG. 16 depicts results describing four major haplotypes observed, with haplotype 2 uniquely defined by two of the associated SNPs, rs2231810 and rs2231805 (R175W). Haplotype 2 was associated with the presence of anti-Cbir1 expression in CD.

FIG. 17 depicts the haplotype block and structure of BTLA. “1” represents the major allele and “2” represents the minor allele.

FIG. 18 depicts a chart describing-the association of BTLA block 1 as associated with Crohn's Disease.

FIG. 19 depicts a chart describing the association of BTLA block 1 H1 with anti-I2 in Crohn's Disease.

FIG. 20 depicts association of BTLA block 1 H3 with small bowel surgery in Crohn's Disease (protective).

FIG. 21 depicts the haplotype block and structure of LIGHT. “1” represents the major allele and “2” represents the minor allele.

FIG. 22 depicts a chart describing the association of LIGHT with anti-I2 expression, for LIGHT block 1 H3.

FIG. 23 depicts a chart describing the association of LIGHT with anti-I2 expression, for LIGHT block 2 H1.

FIG. 24 depicts a chart describing the association of LIGHT block 1 H3 with fibrostenotic disease in Crohn's Disease (protective).

FIG. 25 depicts a chart describing the combined effect of BTLA block 1 H1 and LIGHT block 2 H1 in the expression of anti-I2.

FIG. 26 depicts a table summarizing both LIGHT and BTLA variants associations.

FIG. 27 depicts a table describing confidence intervals for both LIGHT and BTLA variants associations.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3^{rd} ed., Cold Spring Harbor Laboratory Press (Cold Spring Harbor, N.Y. 2001), provide one skilled In the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Haplotype” as used herein, refers to a set of single nucleotide polymorphisms (SNPs) on a gene or chromatid that are statistically associated.

“Risk” as used herein refers to an increase in susceptibility to IBD, including but not limited to CD and UC.

“Protective” and “protection” as used herein refer to a decrease in susceptibility to IBD, including but not limited to CD and UC.

“CD” and “UC” as used herein refer to Crohn's Disease and Ulcerative colitis, respectively.

“SNP” as used herein refers to single nucleotide polymorphism.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

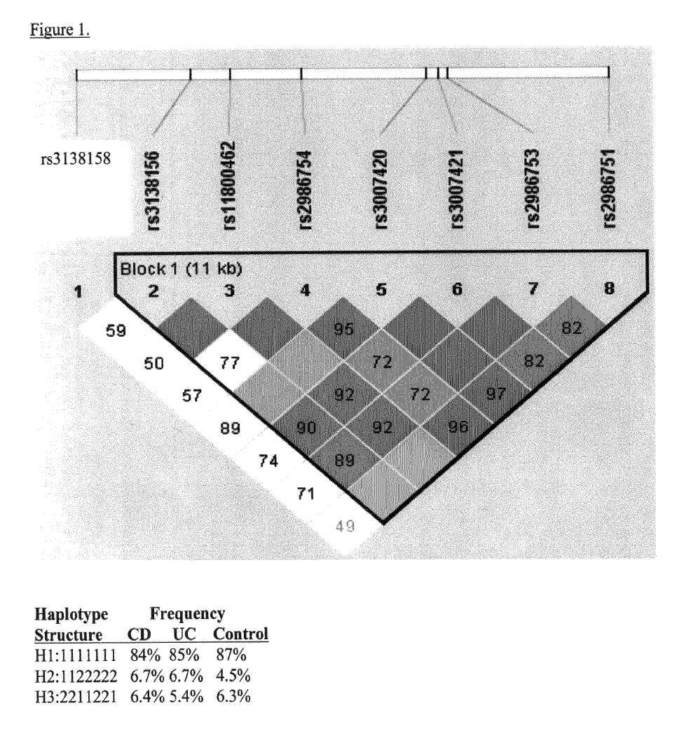

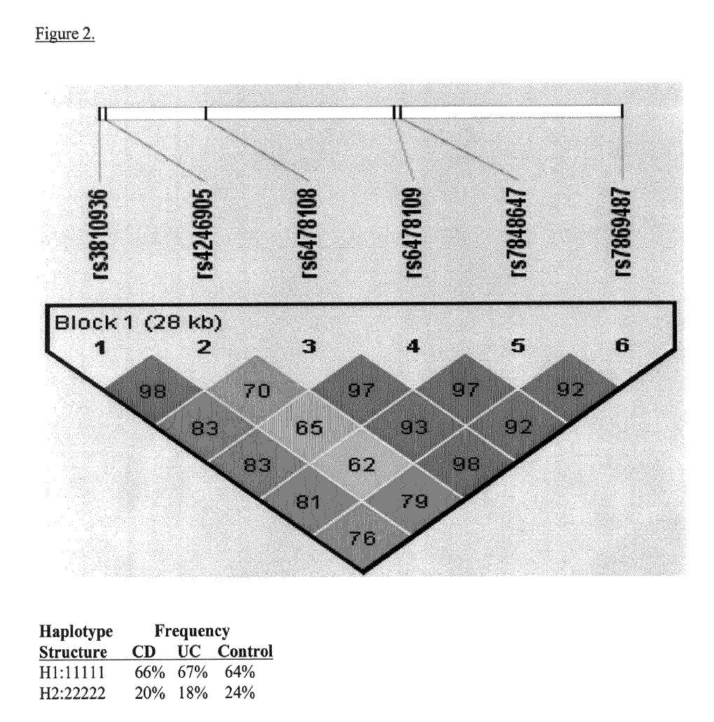

As used herein, DR3 is also known as TNFRSF25. Various examples and versions of homo sapiens nucleotide sequences of DR3 are described herein as SEQ. ID. NO.: 1, SEQ. ID. NO.: 2, SEQ. ID. NO.; 3, SEQ. ID. NO.; 4, SEQ. ID. NO.: 5, and SEQ. ID. NO.: 6. As would be readily apparent to one of skill in the art, these sequences are intended to be illustrative rather than restrictive. As used herein, the terms “haplotype H2 in the DR3 gene” and “haplotype H1 in the DR3 gene” are described in FIG. 1 herein. Rs3138156, rs11800462, rs2986754, rs3007420, rs3007421, rs2986753 and rs2986751 are described herein as SEQ. ID. NO.: 7, SEQ. ID. NO.: 8, SEQ. ID. NO.: 9, SEQ. ID. NO.: 10, SEQ. ID. NO.: 11, SEQ. ID. NO.: 12 and SEQ. ID. NO.: 13, respectively, and are DR3 SNPs used to construct DR3 haplotypes described in FIG. 1.

As used herein, TL1A is also known as TNFSF15. As used herein, the term “haplotype H2 in the TL1A gene” is described in FIG. 2. rs3810936, rs4246905, rs6478108, rs6478109, rs7848647 and rs7869487 are described herein as SEQ. ID. NO.; 14, SEQ. ID. NO.: 15, SEQ. ID. NO.: 16, SEQ. ID. NO.: 17, SEQ. ID. NO.: 18 and SEQ. ID. NO.: 19, respectively, and are TL1A SNPs used to construct TL1A haplotypes described in FIG. 2.

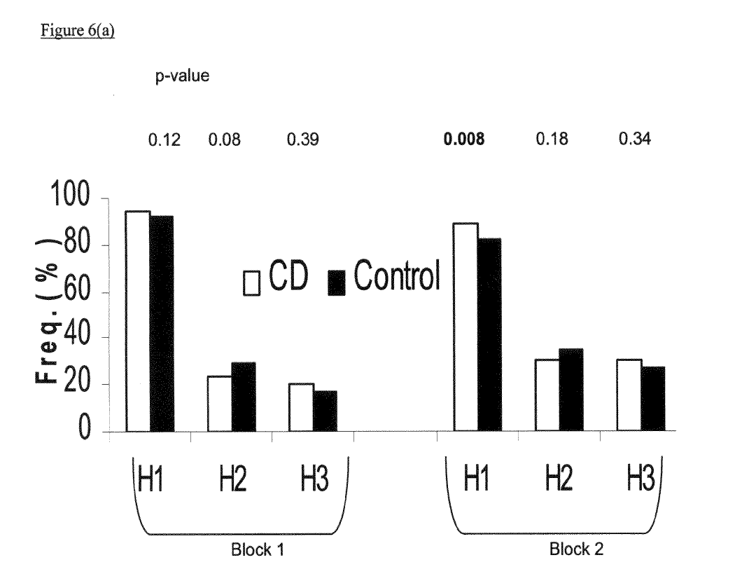

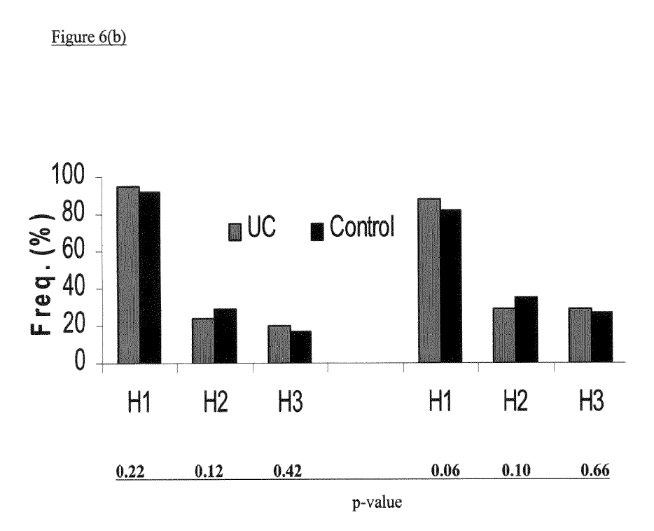

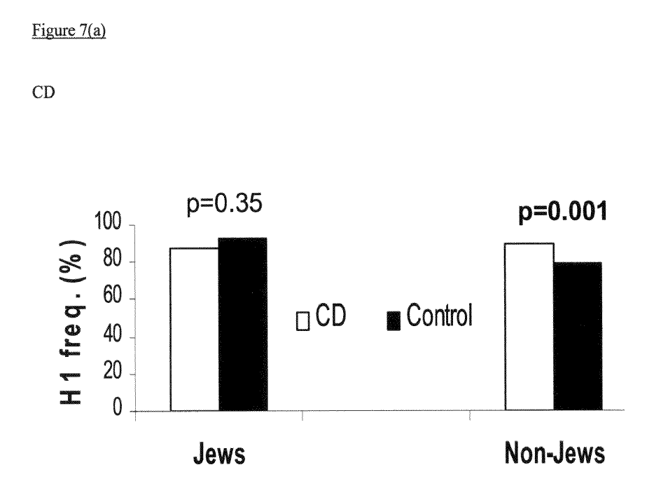

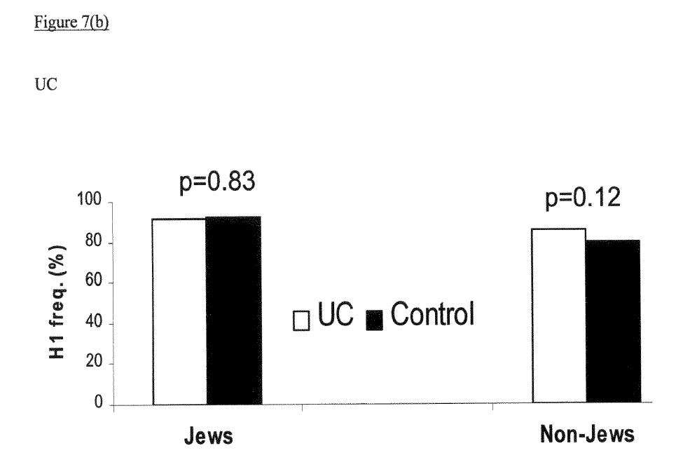

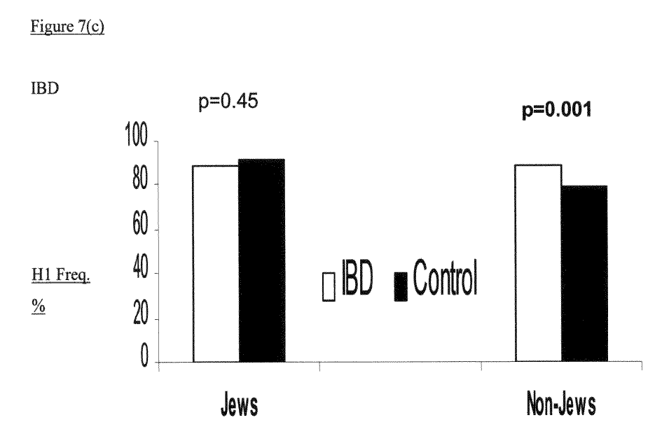

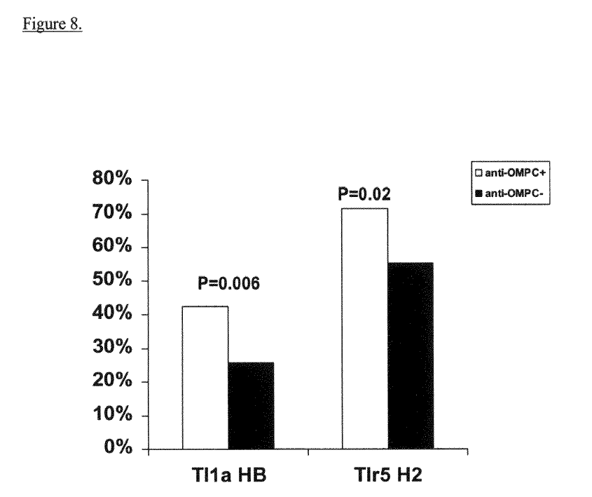

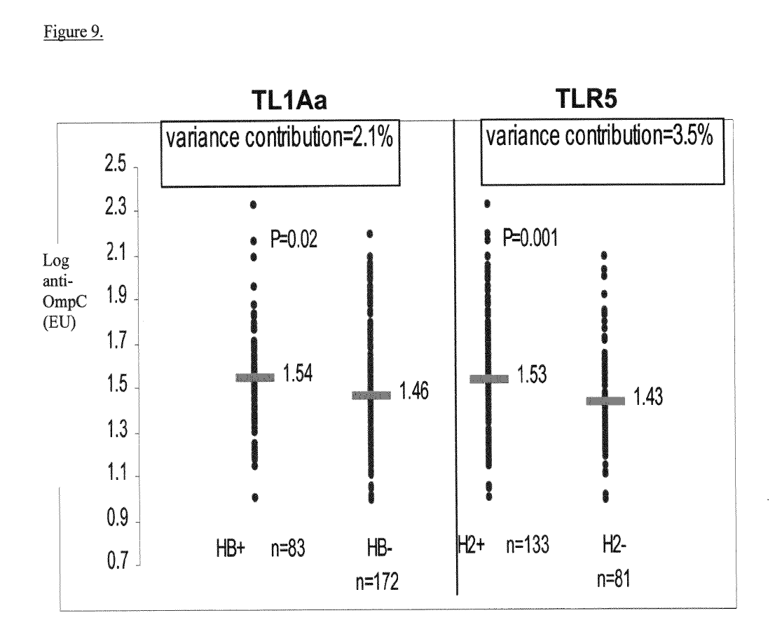

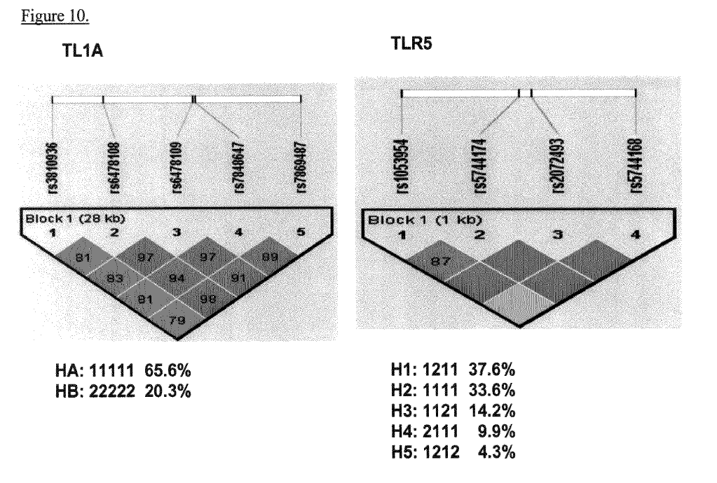

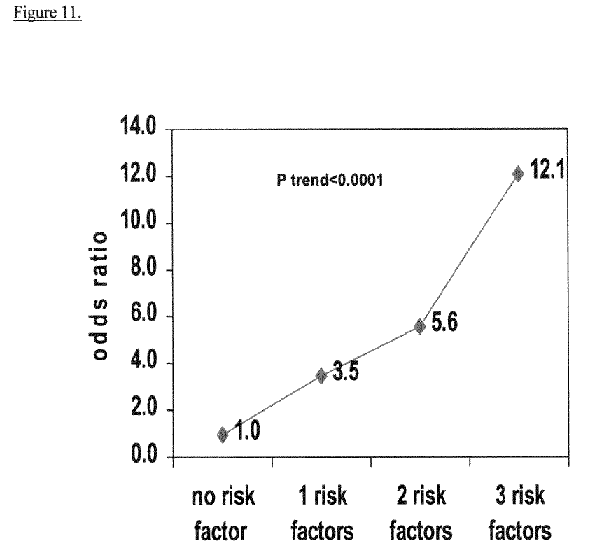

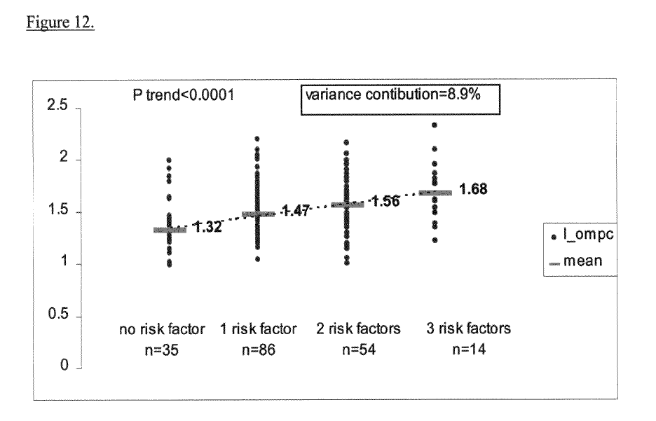

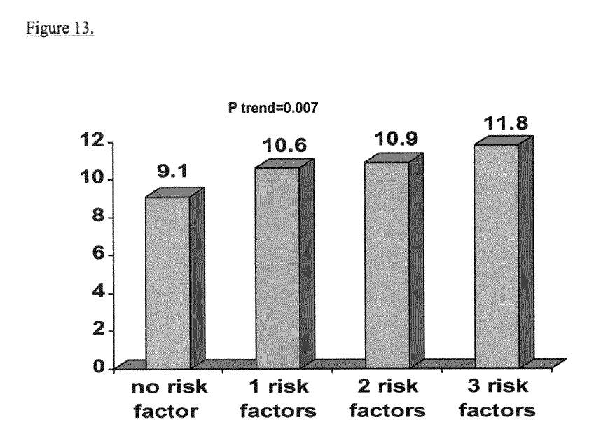

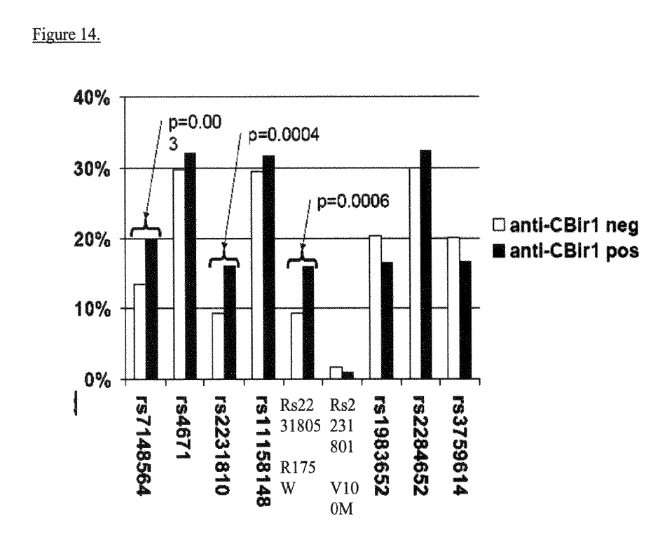

Examples and versions-of homo sapiens nucleotide sequences of GATA3 (GATA binding protein 3) nucleotide sequence are described herein, as SEQ. ID. NO.: 20 and SEQ. ID. NO.: 21. As would be readily apparent to one of skill in the art, these sequences are intended to be illustrative rather than restrictive. As used herein, the term “haplotype Block 2 H1 in the GATA3 gene” is described in FIG. 5. rs1244186, rs1399180, rs570618, rs528778, rs1243963, rs2229360 and rs477461 are described herein, as SEQ. ID. NO.: 22, SEQ. ID. NO.: 23, SEQ. ID. NO.: 24, SEQ. ID. NO.: 25, SEQ. ID. NO.; 26, SEQ. ID. NO.; 27 and SEQ. ID. NO.: 28, respectively, and are GATA3 SNPs used to construct GATA3 haplotypes described in FIG. 5.

As used herein, “TL1A HB,” also known as haplotype B at the TL1A locus, and “TLR5 H2,” also known as haplotype 2 at the TLR5 locus, are described in FIG. 10. rs3810936, rs6478108, rs6478109, rs7848647 and rs7869487 are described herein as SEQ. ID. NO.: 29, SEQ. ID. NO.: 30, SEQ. ID. NO.: 31, SEQ. ID. NO.: 32 and SEQ. ID. NO.: 33, respectively, and are TL1A SNPs used to construct TL1A haplotypes described in FIG. 10. rs1053954, rs5744174, rs2072493 and rs5744168 are described herein as SEQ. IP. NO.: 34, SEQ. ID. NO.: 35, SEQ. ID. NO.: 36 and SEQ. ID. NO.: 37, respectively, and are TLR5 SNPs used to construct TLR5 haplotypes described in FIG. 10.

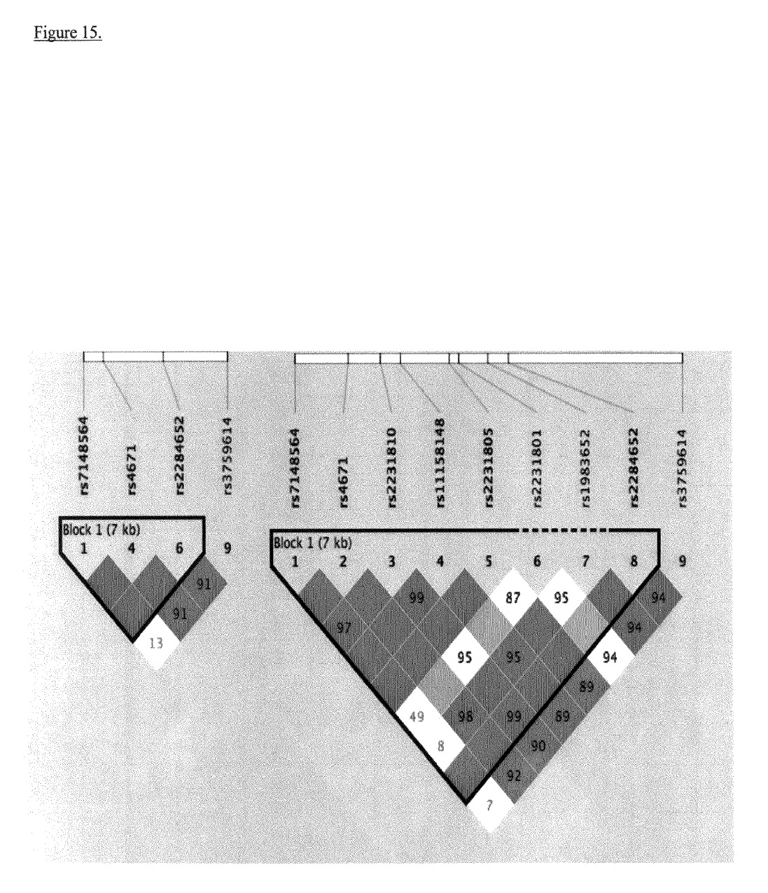

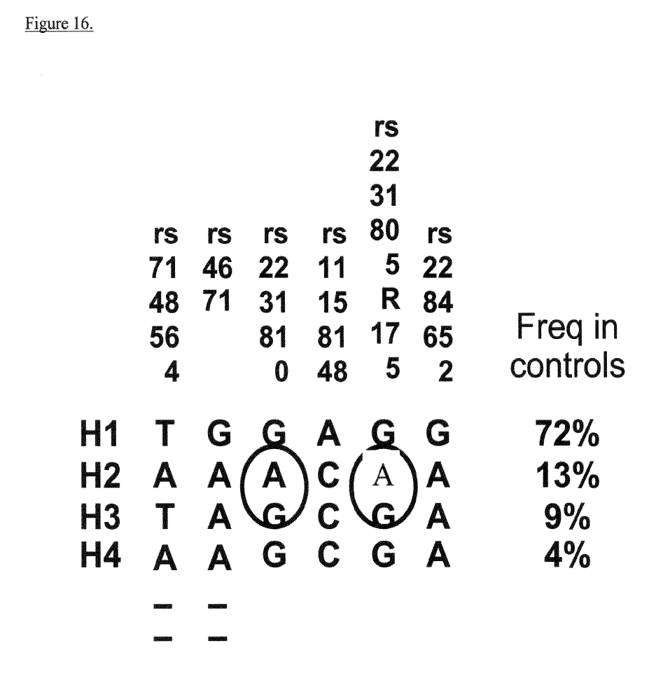

As used herein, “SIN(EFS) H2,” also known as haplotype 2 at the SIN(EFS) locus, is described in FIGS. 15 and 16. rs7148564, rs4671, rs2284652,rs7148564, rs4671, rs2231810, rs11158148, rs2231805 (or R175W), rs2231801, rs1983652, rs2284652 are described herein as SEQ. ID. NO.: 38, SEQ. ID. NO.: 39; SEQ. ID. NO.: 40, SEQ. ID. NO.: 41, SEQ. ID. NO.: 42, SEQ. ID. NO.: 43, SEQ. ID. NO.: 44, SEQ. ID. NO. 45, SEQ. ID. NO.: 46, SEQ. ID. NO.: 47 and SEQ. ID. NO.: 48, respectively, and are SIN(EFS) SNPs used to construct SIN(EFS) haplotypes described in FIGS. 15 and 16.

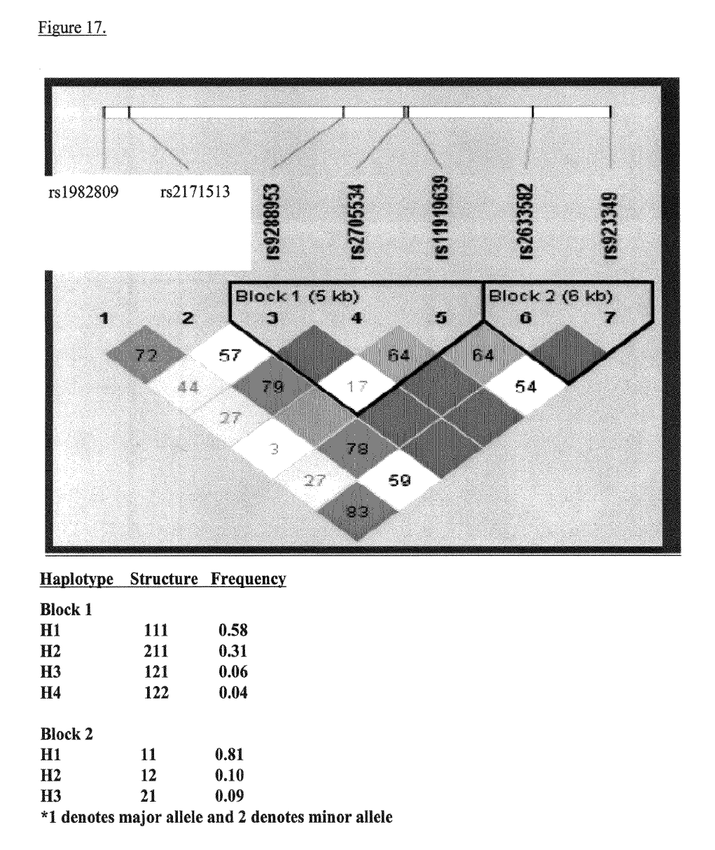

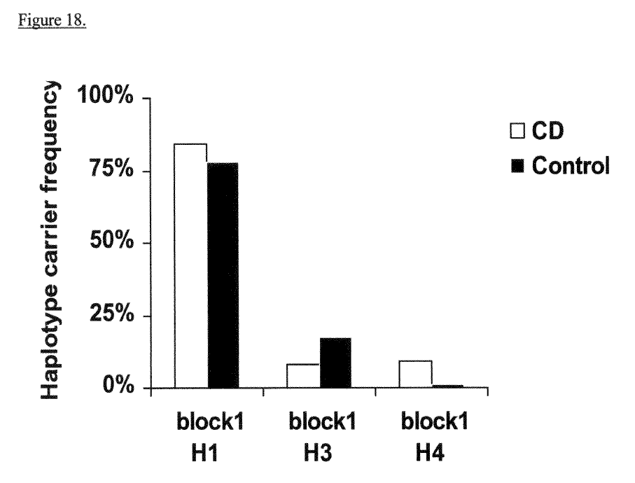

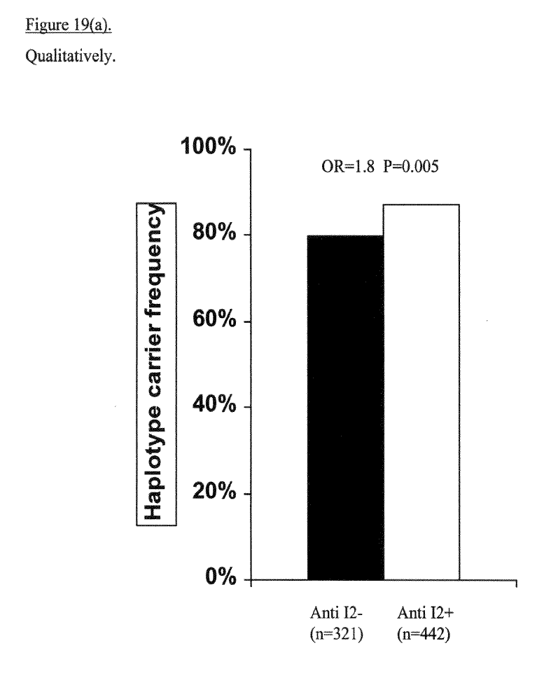

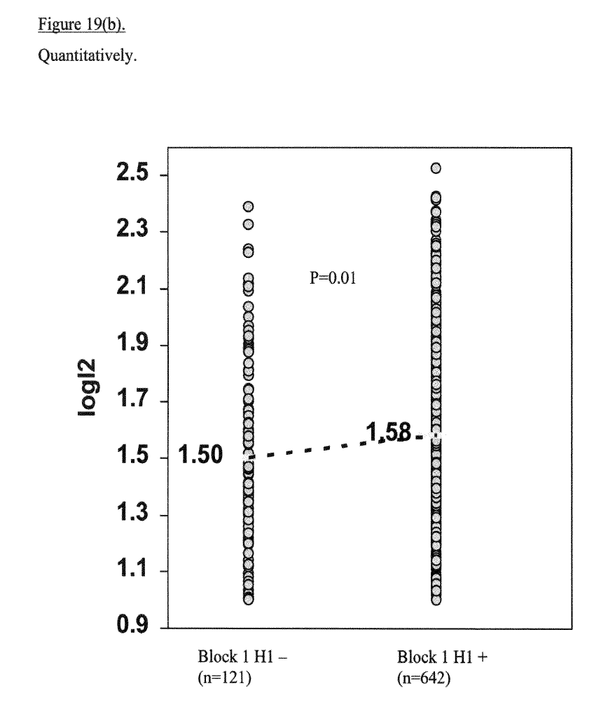

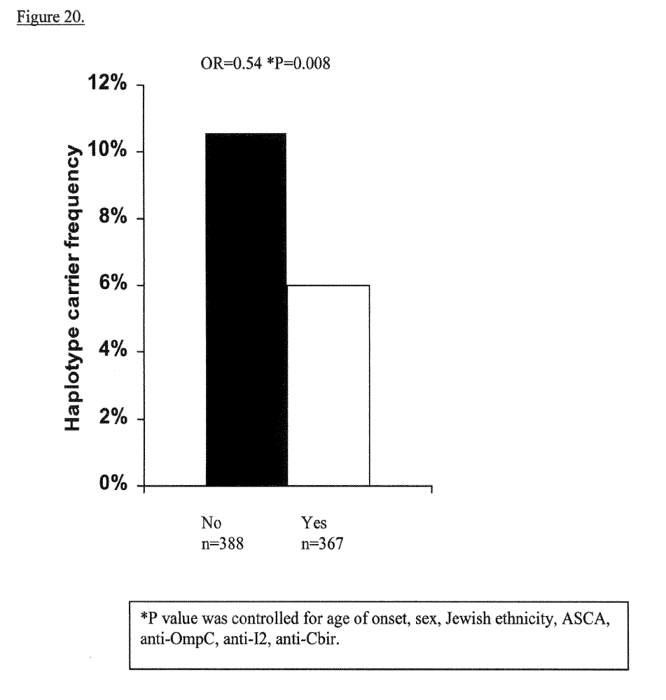

As used herein, the term “BTLA” is an abbreviation for B and T lymphocyte annenuator. BTLA B1 H1, also known as BTLA Block 1. Haplotype 1, and BTLA B1 H3, also known as BTLA. Block 1 Haplotype 3, are described in FIG. 17. rs9288953, rs2705534, rs11919639, rs2633582 and rs923349 are described herein as SEQ. ID. NO.: 49, SEQ. ID. NO.: 50, SEQ. ID. NO.: 51, SEQ. ID. NO.: 52 and SEQ. ID. NO.: 53, respectively, and are BTLA SNPs used to construct BTLA haplotypes described in FIG. 17.

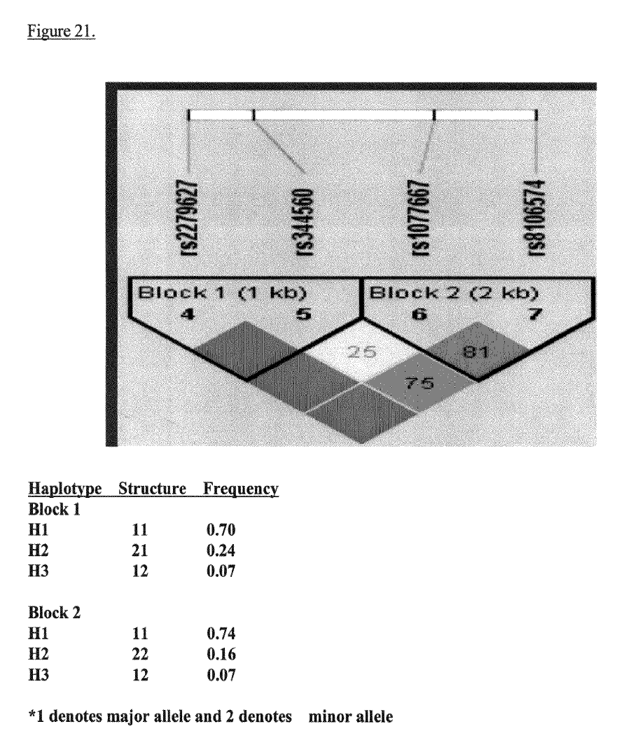

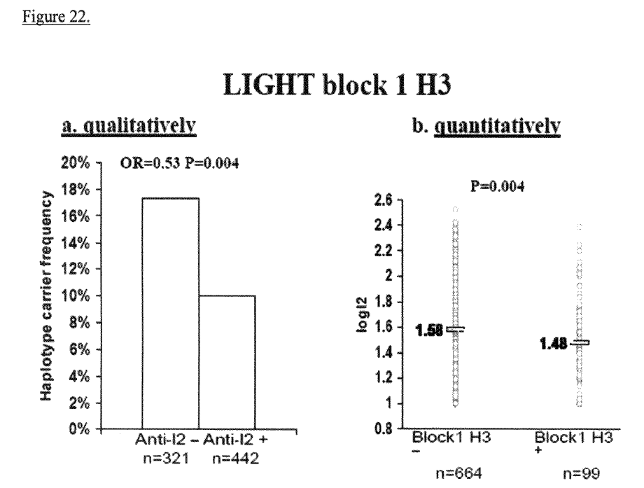

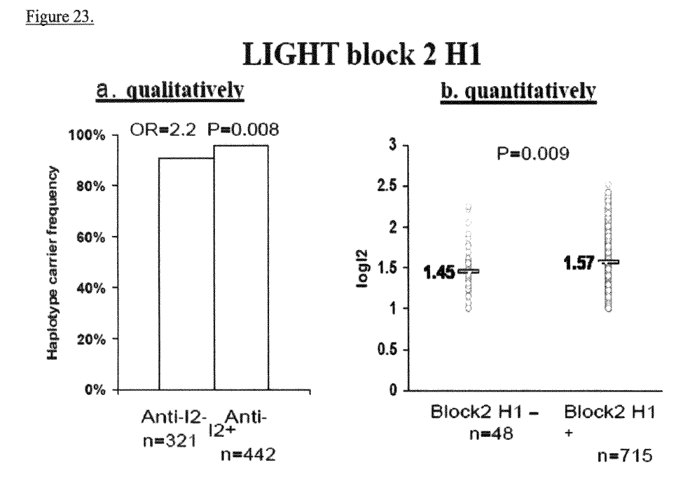

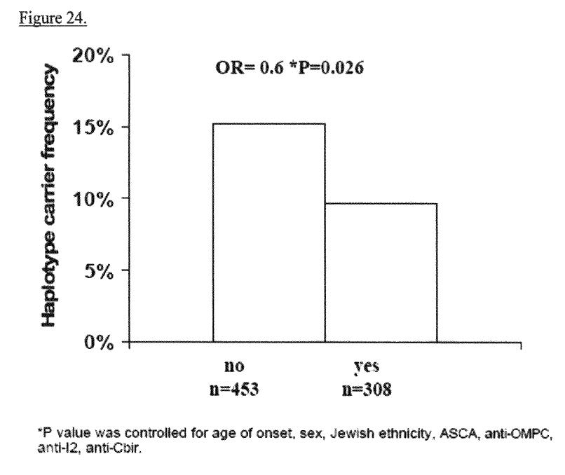

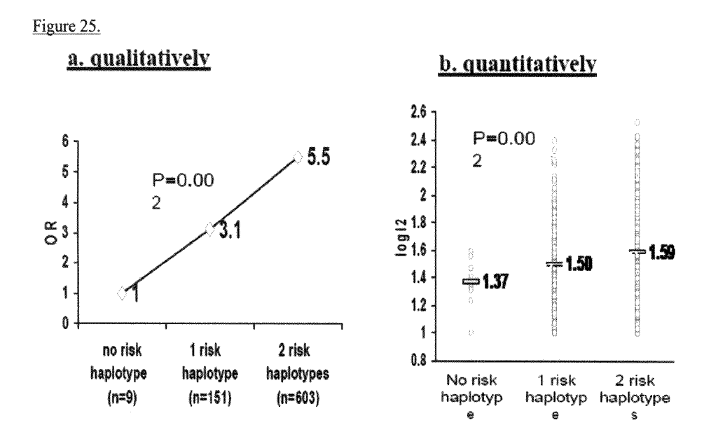

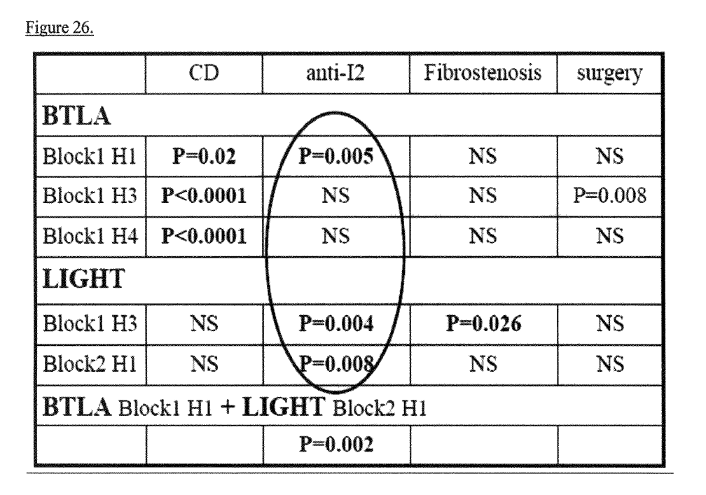

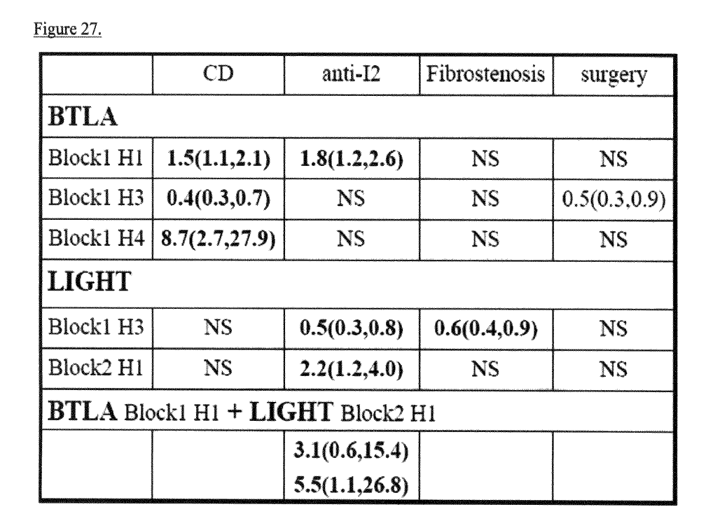

As used herein, the term “LIGHT” is an abbreviation for TNF receptor superfamily 14. LIGHT B1 H3, also known as LIGHT Block 1 Haplotype 3, and LIGHT B2 H3, also known as LIGHT Block 2 Haplotype 3, are described in FIG. 21. rs2279627, rs344560, rs1077667 and rs8106574 are described herein as SEQ. ID. NO.: 54, SEQ. ID. NO.: 55, SEQ. ID. NO.: 56 and SEQ. ID. NO.: 57, respectively, and are LIGHT SNPs used to construct LIGHT haplotypes described in FIG. 21.

The inventors performed a genome-wide association study testing autosomal, single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to CD. These SNPs and haplotypes are suitable for genetic testing to identify at risk individuals and those with increased risk for complications associated with serum expression of Anti-Saccharomyces cerevisiae antibody, and antibodies to 12, OmpC, and Cbir. The detection of protective and risk SNPs and/or haplotypes may be used to identify at risk individuals predict disease course and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease and ulcerative colitis. Other embodiments provide for methods of prognosing inflammatory bowel disease including but not limited to Crohn's Disease and ulcerative colitis. Other embodiments provide for methods of treating inflammatory bowel disease including but not limited to Crohn's Disease and ulcerative colitis.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease and ulcerative colitis, as described herein. The methods may also further include recording whether a genetic risk, susceptibility for inflammatory bowel disease including but not limited to Crohn's Disease and ulcerative colitis exists in the individual. The methods may also further include a prognosis of inflammatory bowel disease based upon the presence or absence-of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced, with tissue obtained from an individual such as tissue obtained during surgery or biopsy procedures.

### I. TL1A/DR3 Variants

As disclosed herein, the inventors investigated whether a genetic interaction between TL1A and DR3 contributed to CD. Eight DR3 and five TL1A SNPs were genotyped in 763 CD, 351 ulcerative colitis (UC) and 254 controls, Haplotype blocks were constructed by Haploview; individual haplotypes were assigned by PHASE and ordered by frequency; associations were tested by chi-square and permutation. Gene-gene interaction was tested by logistic regression.

As further disclosed herein, two major haplotypes of DR3 were found to be associated with CD. In non-Jews, CD patients had a lower frequency of homozygotes of H1 (66.2% vs. 76.7%, p=0.007) and a higher frequency of H2 carriers (13.1% vs. 7.5%, p=0.035) when compared with controls; however, this association was absent in Jewish CD. In non-Jewish UC, a similar trend of association for H1 and H2 was also observed. H2 of TL1A has been reported to be negatively associated with CD (39% vs. 50%) and UC (37.3% vs. 50%), and this effect was also seen only in non-Jews. When analyzing DR3 and TL1A together, a significant dose-effect was observed among protective factors (DR3 H1 and TL1A H2) in non-Jewish IBD (p trend<0.0001), odds ratio ranging from 1 to 0.47 (1 protective factor) to 0.19 (both protective factors). No statistical interaction was detected between these two genes. The DR3 association observed shows that the TL1A/DR3 interaction contributes to CD pathogenesis. “Hits” from genome-wide association studies will identify additional pathways that contain other genetic determinants of complex traits.

In one embodiment, the present invention provides methods of diagnosing and/or predicting susceptibility to IBD in an individual by determining the presence or absence m the individual of haplotype H2 in the DR3 gene. In another embodiment, the present invention provides methods of prognosis of IBD in an individual by determining the presence or absence in the individual of haplotype H2 in the DR3 gene. In another embodiment, the present invention provides methods of diagnosing and/or predicting susceptibility to Crohn's Disease. In another embodiment, the present invention provides methods of diagnosing and/or predicting susceptibility to Crohn's Disease in a non-Jewish individual.

In another embodiment, the present invention provides methods of treatment of IBD in an individual by inhibiting the expression of haplotype H2 in the DR3 gene.

In one embodiment, the present invention provides methods of diagnosing and/or predicting protection against IBD in an individual by determining the presence or absence in the individual of haplotype H1 in the DR3 gene. In another embodiment, the present invention provides methods of prognosis of IBD in an individual by determining the presence or absence in the individual of haplotype H1 in the DR3 gene. In another embodiment, the present invention provides methods of diagnosing and/or predicting protection against Crohn's Disease. In another embodiment, the present invention provides methods of diagnosing and/or predicting protection against Crohn's Disease in anon-Jewish individual.

In one embodiment, the present invention provides methods of diagnosing and/or predicting protection against IBD in an individual by determining the presence or absence in the individual of haplotype H2 in the TL1A gene. In another embodiment, the present invention provides methods of prognosis of IBD in an individual by determining the presence or absence in the individual of haplotype H2 in the TL1A gene. In another embodiment, the present invention provides methods of diagnosing and/or predicting protection against Crohn's Disease. In another embodiment, the present invention provides methods of diagnosing and/or predicting protection against Crohn's Disease in a non-Jewish individual.

### II. GATA3 Variants

As disclosed herein, the inventors tested the association of GATA3 variation with CD and UC. Seven GATA3 SNPs were genotyped in 763 CD, 351 UC and 254 controls; haplotype blocks were constructed by using haploview 3.3; haplotypes were assigned using: PHASE 2.0; association tests were performed by using chi-square. Two haplotype blocks with 3 haplotypes per block (freq>0.05) were observed. Block 2, haplotype 1 (H1:1111) was associated with CD (88.7% CD vs 82.3% controls, OR=1.7, 95% CI: 1.14-2.51, p=0.008). H1 was associated with UC with borderline statistical significance (87.7% UC, 82.3% control, p=0.06). This association was strongest in non-Jews (89.5% CD, 79.6% control p=0.001).

As further disclosed herein, the observation of an association between haplotype in GATA3 and CD shows that. GATA3 variation contributes to CD pathogenesis through possible effects on Th1/Th2 dysregulation.

In one embodiment, the present invention provides methods of diagnosing and/or predicting susceptibility to IBD in an individual by determining the presence or absence in the individual of haplotype Block 2 H1 in the GATA3 gene. In another embodiment, the present invention provides methods of prognosis of IBD in an individual by determining the presence or absence in the individual of haplotype Block 2 H1 in the GATA3 gene. In another embodiment, the present invention provides methods of diagnosing and/or predicting susceptibility to Crohn's Disease. In another embodiment, the present invention provides methods of diagnosing and/or predicting susceptibility to Crohn's Disease in a non-Jewish individual. In another embodiment, susceptibility to IBD is determined in conjunction with the presence of Th1/Th2 dysregulation.

In another embodiment, the present invention provides methods of treating IBD in an individual by inhibiting expression of haplotype Block 2 H1 in the GATA3 gene. In another embodiment, the present invention provides methods of treating CD in an individual by inhibiting expression of haplotype Block 2 H1 in the GATA3 gene. In another embodiment, the present invention provides methods of treating Th1/Th2 dysregulation in an individual by inhibiting the expression of haplotype Block 2 H1 in the GATA3 gene. In another embodiment, the present invention provides methods of treating IBD in an individual by determining the presence or absence of haplotype Block 2 H1 in the GATA3 gene and then inhibiting the activation of T-cell receptor gene.

### III. TL1A, TLR5 and NOD2 Variants and OmpC

As disclosed herein, the inventors found TL1A HB and TLR5 H2 was positively associated with anti-OmpC expression in Jews only, NOD2 by itself was not associated with anti-OmpC in Jews. However, the risk haplotypes or variants from these 3 genes combined increased anti-OmpC expression. In Jews, the sum by quartile of the expression of all antibodies also increased with, increasing number of genetic risk factors. The additive effect among TL1A, TLR5 and NOD2 in the expression of anti-OmpC and antibody quartile sum was not observed in the non-Jewish CD population. In Jewish CD subjects, TL1A HB, TLR5 H2, and NOD2 mutations are additive for increased expression of anti-OmpC.

In one embodiment, the present invention provides a method of diagnosing susceptibility to a Crohn's Disease subtype in an individual by determining the presence or absence of a high magnitude of anti-OmpC expression relative to a healthy individual and the presence or absence of a high magnitude of anti-OmpC expression relative to a healthy individual, where the presence of a high magnitude of anti-OmpC expression relative to a healthy individual and the presence of one or more risk variants selected from the group consisting of haplotype B at the TL1A locus, haplotype 2 at the TLR5 locus, and/or CD-associated variants at the NOD2 locus, is indicative of susceptibility to the Crohn's Disease subtype. In another embodiment, the risk variants are additive for an increased expression of anti-OmpC. In another embodiment, the invention provides a method of treating a Crohn's Disease subtype in an individual by determining the presence of a high magnitude of anti-OmpC expression relative to a healthy individual and the presence of one or more risk variants selected from the group consisting of haplotype B at the TL1A locus, haplotype 2 at the TLR5 locus, and/or CD-associated variants at the NOD2 locus, and treating the Crohn's Disease subtype. In another embodiment, the individual is Jewish.

### IV. SIN(EFS) Variants and Cbir1

As disclosed herein, the inventors tested the association of the SIN(EFS) gene with CD and expression of anti-CBir1 antibody. SIN(EFS) haplotype 2 was found to be associated with anti-CBir1 expression in human CD as was the non-synonymous SNP R175W variant.

In one embodiment, the present invention provides a method of diagnosing susceptibility to a Crohn's Disease subtype in an individual by determining the presence or absence of a high magnitude of anti-Cbir1 expression relative to a healthy individual and the presence or absence of a risk haplotype at the SIN(EFS) locus, where the presence of a high magnitude of anti-Cbir1 expression and/or the presence of a risk haplotype at the SIN(EFS) locus is indicative of susceptibility to the Crohn's Disease subtype. In another embodiment, the risk haplotype is haplotype 2 at the SIN(EFS) locus. In another embodiment, the risk haplotype comprises variant R175W. In another embodiment, the present invention provides a method of treating a Crohn's Disease subtype in an individual by determining the presence of a high magnitude of anti-Cbir1 expression and/or the presence of a risk haplotype at the SIN(EFS) locus and treating the Crohn's Disease subtype.

### V. BTLA and LIGHT Variants

As disclosed herein, the inventors found BTLA and LIGHT acted as a molecular switch in modulating T cell activation. The inventors found BTLA block1 to be associated with CD. Specifically, BTLA block1 H1 was associated with anti-I2 expression in CD, BTLA block1 H3 was associated with small bowel surgery in CD (protective). LIGHT was found to be associated with anti-I2 expression, specifically LIGHT block 1 H3 and LIGHT block 2 H1. LIGHT Block 1 B3 was found to be associated with fibrostenotic disease in CD (protective).

In one embodiment, the present invention provides a method of diagnosing susceptibility to Crohn's Disease in an individual by determining the presence or absence of Block 1 haplotype 4 at the BTLA locus, where the presence of Block 1 haplotype 4 at the BTLA locus is indicative of susceptibility to Crohn's Disease. In another embodiment, the present invention provides a method of treating Crohn's Disease by determining the presence of Block 1 haplotype 4 at the BTLA locus and treating the Crohn's Disease.

In one embodiment, the present invention provides a method of diagnosing susceptibility to a Crohn's Disease subtype in an individual by determining the presence or absence of a high magnitude, of anti-I2 expression relative to a healthy individual and the presence or absence of Block 1 haplotype 1 at the BTLA locus and/or Block 2 haplotype 1 at the LIGHT locus, where the presence of a high magnitude of anti-I2 expression relative to a healthy individual and the presence of Block 1 haplotype 1 at the BTLA locus and/or Block 2 haplotype 1 at the LIGHT locus is indicative of susceptibility to the Crohn's Disease subtype. In another embodiment, the present invention provides a method of treating a Crohn's Disease subtype in an individual by determining the presence of a high magnitude of anti-I2 expression relative to a healthy individual and the presence of Block 1 haplotype 1 at the BTLA locus and/or Block 2 haplotype 1 at the LIGHT locus, and treating the Crohn's Disease subtype.

In one embodiment, the present invention provides a method of diagnosing protection against Crohn's Disease in an individual by determining the presence or absence of Block 1 haplotype 3 at the BTLA locus, where the presence of Block 1 haplotype 3 at the BTLA locus is indicative of a decreased likelihood of Crohn's Disease relative to a healthy individual. In another embodiment, the presence of Block 1 haplotype 3 is indicative of a decreased likelihood of the small bowel surgery Crohn's Disease subtype relative to a healthy individual.

In one embodiment, the present invention provides a method of diagnosing protection against Crohn's Disease in an individual by determining the presence or absence of a high magnitude of anti-I2 expression and the presence or absence of Block 1 haplotype 3 at the LIGHT locus, where the absence of a high magnitude of anti-I2 expression and the presence of Block 1 haplotype 3 at the LIGHT locus is indicative of a decreased likelihood of Crohn's Disease relative to a healthy individual. In another embodiment, the absence of a high magnitude of anti-I2 expression and the presence of Block 1 haplotype 3 at the LIGHT locus is indicative of a decreased likelihood of the fibrostenotic Crohn's Disease subtype relative to a healthy individual.

### VI. MAGI2 Variants

As disclosed herein, the inventors investigated the role genetic variants in the tight junction pathway gene MAGI2 may have in the development of Crohn's Disease and ulcerative: colitis. Using a haplotype tagging approach, the Inventors identified association between variants within MAGI2 and IBD, UC and CD, including various risk alleles, as listed in full in Table 3 herein. A three marker intron 2 MAGI2 haplotype (rs7785088, rs323149 and rs13246026) was protective against IBD susceptibility (IBD haplotype frequency 8.2% vs, control haplotype frequency 12.9%, OR 0.61 (95% CI 0.44-0.86), p=0.006). Crohn's Disease was also associated with alleles in introns 2, 5, 6 and 20, An intron 6 SNP (rs2160322) was significantly associated with ulcerative, colitis with the common C allele being the risk allele (UC 72.2% vs control allele frequency 63.6% (OR 1.49 (1.12-1.97), (p=0.006)). Two further SNPs (rs7788384 and rs2110871) and a 2 marker haplotype (rs7803276/rs7803705) from intron 6 were associated with ulcerative colitis susceptibility. Intron 20 (p=0.03) and intron 2 (p=0.02) SNPs were also associated with ulcerative colitis.

In one embodiment, the present invention provides a method of diagnosing and/or predicting susceptibility to inflammatory bowel disease by determining the presence or absence of a risk haplotype and/or variant at the MAGI2 locus, where the presence of the risk haplotype and/or variant at the MAGI2 locus is indicative, of susceptibility to inflammatory bowel disease. In another embodiment, the present invention provides a method of treating inflammatory bowel disease by determining the presence of a risk haplotype and/or variant at the MAGI2 locos and treating the inflammatory bowel disease. In another embodiment, the inflammatory bowel disease is Crohn's Disease. In another embodiment, the inflammatory bowel disease is ulcerative colitis.

In another embodiment, the present invention provides a method of diagnosing and/or predicting protection against inflammatory bowel disease by determining the presence or absence of a protective haplotype at the MAGI2 locus, where the presence of the protective haplotype at the MAGI2 locus is indicative of a decreased likelihood of susceptibility to inflammatory bowel disease relative to a healthy individual. In another embodiment, the present invention provides a method of diagnosing and/or predicting protection against inflammatory bowel disease by determining the presence or absence of a protective variant at the MAGI2 locus, where the presence of the protective variant at the MAGI2 locus is indicative of a decreased likelihood of susceptibility to inflammatory bowel disease relative to a healthy individual. In another embodiment, the inflammatory bowel disease is Crohn's Disease. In another embodiment, the inflammatory bowel disease is ulcerative colitis.

In one embodiment, the present invention provides a method of determining susceptibility to an inflammatory bowel disease subtype by determining the presence or absence of a risk variant at the MAGI2 locus and the presence or absence of a high magnitude of anti-ASCA expression relative to a healthy individual, where the presence of the risk variant at the MAGI2 locus and/or the presence of the high magnitude of anti-ASCA expression relative to a healthy individual is indicative of susceptibility to the inflammatory bowel disease subtype. In another embodiment, the present invention provides a method of treatment of an inflammatory bowel disease subtype by determining the presence of a risk variant at the MAGI2 locus and/or the presence of a high magnitude of anti-ASCA expression relative to a healthy individual, and treating the inflammatory bowel disease subtype.

In one embodiment, the present invention provides a method of determining susceptibility to an inflammatory bowel disease subtype by determining the presence or absence of a risk variant at the MAGI2 locus and the presence or absence of a high magnitude of anti-Cbir1 expression relative to a healthy individual, where the presence of the risk variant at the MAGI2 locus and/or the presence of the high magnitude of anti-Cbir1 expression relative to a healthy individual is indicative of susceptibility to the inflammatory bowel disease subtype. In another embodiment, the present invention provides a method of treatment of an inflammatory bowel disease subtype by determining the presence of a risk variant at the MAGI2 locus and/or the presence of a high magnitude of anti-Cbir1 expression relative to a healthy individual, and treating the inflammatory bowel disease subtype.

In one embodiment, the present invention provides a method of determining susceptibility to an inflammatory bowel disease subtype by determining the presence or absence of a risk variant, at the MAGI2 locus and the presence or absence of a high, magnitude of anti-OmpC expression relative to a healthy individual, where the presence of the risk variant at the MAGI2 locus and/or the presence of the high magnitude of anti-OmpC expression relative to a healthy individual is indicative of susceptibility to the inflammatory bowel disease subtype. In another embodiment, the present invention provides a method of treatment of an inflammatory bowel disease subtype by determining the presence of a risk variant at the MAGI2 locus and/or the presence of a high magnitude of anti-OmpC expression relative to a healthy individual, and treating the inflammatory bowel disease subtype.

In one embodiment, the present invention, provides a method of determining protection against an inflammatory bowel disease subtype in an individual by determining the presence or absence of a protective haplotype at the MAGI2 locus and the presence or absence of a high magnitude of expression relative to a healthy individual of anti-ASCA, anti-Cbir1 and/or anti-OmpC, where the presence of a protective haplotype at the MAGI2 locus and the presence of a high magnitude of expression relative to a healthy individual of anti-ASCA, anti-Cbir1 and/or anti-OmpC is indicative of a decreased likelihood of the inflammatory bowel, disease subtype.

### VII. Variety of Methods and Materials

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis, et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant, energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present m the sample. Mismatches between, a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove hinder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in. Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect, mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In. DGGE, double-stranded DNA is electrophoresed in a gel containing an increasing concentration of denaturant; double-stranded fragments-made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art, and useful in the methods, of the invention. Other well-known approaches, for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against inflammatory Bowel Disease in an individual may be practiced using one or any combination of the well known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended, to limit the invention. One skilled in the art may develop equivalent means or reactants without the exercise of inventive capacity and without departing from the scope of the invention.

### Example 1

### Association Between IBD and the TL1A/DR3 Ligand/Recepior Pair

The inventors investigated whether a genetic interaction between TL1A and DR3 contributed to CD. Eight DR3 and five TL1A SNPs were genotyped in 763 CD, 351 ulcerative colitis (UC) and 254 controls. Haplotype blocks were constructed by Haploview; individual haplotypes were assigned by PHASE and ordered by frequency; associations were tested by chi-square and permutation. Gene-gene interaction was tested by logistic regression.

Two major haplotypes of DR3 were found to be associated with CD. In non-Jews, CD patients had a lower frequency of homozygotes of H1 (66.2% vs. 76.7%, p=0.007) and a higher frequency of H2 carriers (13.1% vs. 7.5%, p=0.035) when compared with controls; however, this association was absent in Jewish CD. In non-Jewish UC, a similar trend of association for H1 and H2 was also observed. H2 of TL1A has been reported to be negatively associated with CD (39% vs. 30%) and UC (37.3% vs. 50%), and this effect was also seen only in non-Jews. When analyzing DR3 and TL1A together, a significant dose-effect was observed among protective factors (DR3 H1 and TL1A H2) in non-Jewish IBD (p trend<0.0001), odds ratio ranging from 1 to 0.47 (1 protective factor) to 0.19 (both protective factors). No statistical interaction was detected between these two genes. The DR3 association observed shows that the TL1A/DR3 interaction contributes to CD pathogenesis. “Hits” from genome-wide association studies will identify additional pathways that contain other genetic determinants of complex traits.

### Example 2

### Association Between IBD and the TL1A/DR3 Ligand/Receptor Pair: Methods

Eight DR3 and 5 TL1A SNPs were genotyped in 763 CD (314 Jews), 351 UC (136 Jews) and 254 controls (51 Jews), DR3 SNPs are described herein as SEQ. ID. NO.: 7, SEQ. ID. NO.: 8, SEQ. ID. NO.: 9, SEQ. ID. NO.: 10, SEQ. ID. NO.; 11, SEQ. ID. NO.: 12 and SEQ. ID. NO.: 13. TL1A SNPs are described herein as SEQ. ID. NO.: 14, SEQ. ID. NO.: 15, SEQ. ID. NO.: 16, SEQ. ID. NO.: 17, SEQ. ID. NO.: 18 and SEQ. ID. NO.: 19. Haplotype blocks were constructed by Haploview; individual haplotypes were assigned by PHASE; associations were tested by chi-square and permutation. Gene-gene interaction was tested by logistic regression.

### Example 3

Association Between IBD and the TL1A/DR3 Ligand/Receptor Pair: Results

Two major haplotypes of DR3 were associated with CD. In non-Jews, CD patients had a lower frequency of homozygotes of H1 (66.2% vs. 76.7%, p=0.007) and a higher frequency of H2 carriers (1.3.1% vs. 7.5%, p=0.035) when compared with controls; however, this association was absent in Jewish CD. In non-Jewish UC, a similar trend of association for H1 and H2 was also observed.

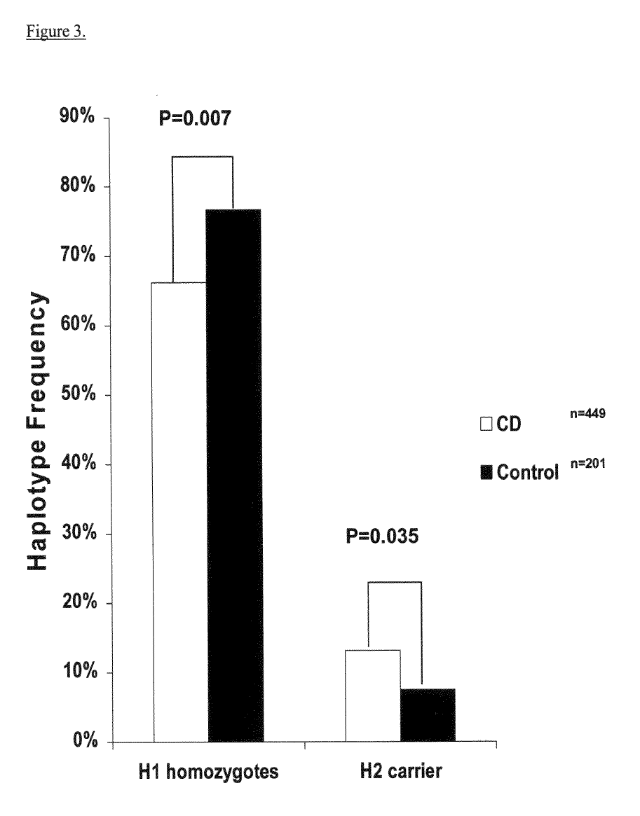

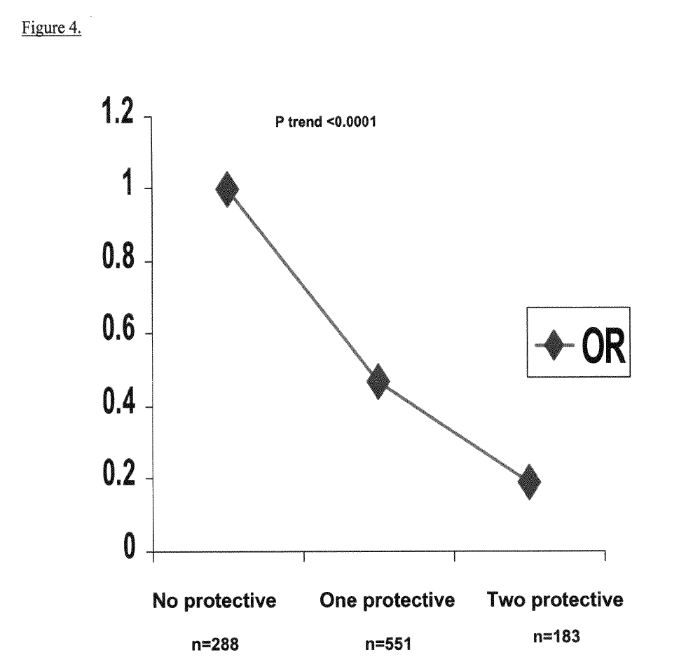

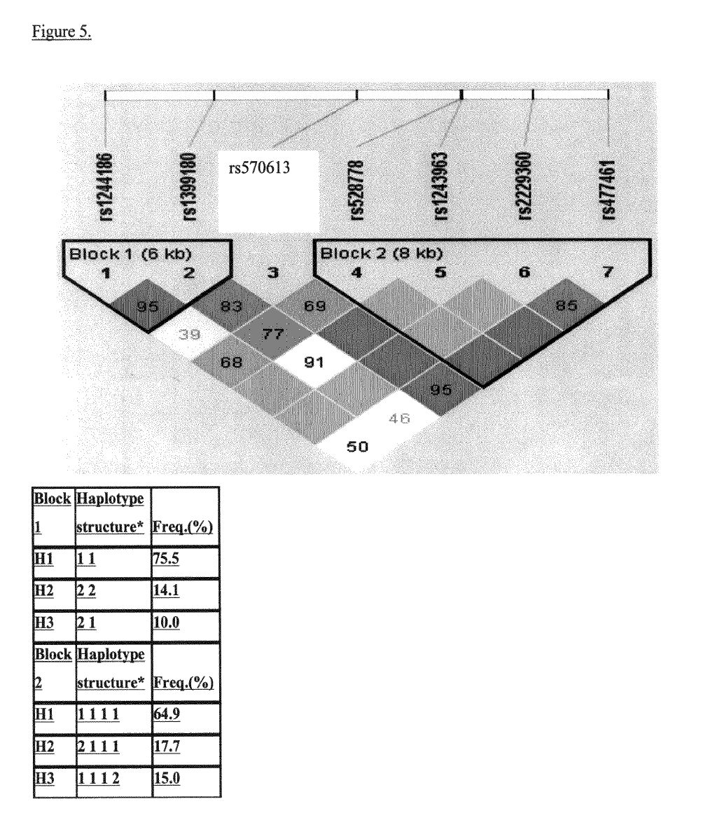

H2 of TL1A has been previously reported to be negatively associated with CD (39% vs. 50%) and UC (37.3% vs. 50%), and this effect was also seen only in non-Jews. When analyzing DR3 and TL1A together, a significant dose-effect, was observed among protective factors (DR3 H1 homozygotes and TL1A H2) in non-Jewish IBD (p trend<0.0001), odds ratio ranging from 1 to 0.47 (1 protective factor) to 0.19 (both protective factors) (FIG. 3). No statistical interaction was detected between these two genes.

### Example 4

### Association Between IBD and the TL1A/DR3 Ligand/Receptor Pair: Conclusions

The DR3 association observed supports the idea that the TL1A/DR3 contributes to CD pathogenesis independently and simultaneously. “Hits” from genome-wide association studies will help to identify other genetic determinants that are involved in the specific pathway.

### Example 5

Association Between a Haplotype of the GATA3 Gene and Inflammatory Bowel Disease

The inventors tested the association of GATA3 variation with CD and UC. Seven GATA3 SNPs were genotyped in 763 CD, 351 UC and 254 controls; haplotype blocks were constructed by using haploview 3.3; haplotypes were assigned using PHASE 2.0; association tests were performed by using chi-square. GATA3 SNPs are described herein as SEQ. ID. NO.: 22, SEQ. ID. NO.: 23, SEQ. ID. NO.; 24, SEQ. ID. NO.; 25, SEQ. ID. NO.: 26, SEQ. ID. NO.: 27 and SEQ. ID. NO.: 28. Two haplotype blocks with 3 haplotypes per block (freq>0.05) were observed. Block 2, haplotype 1 (H1:1111) was associated with CD (88.7% CD vs 82.3% controls, OR=1.7, 95% CI: 1.14-2.51, p=0.008). H1 was associated with UC with borderline statistical significance (87.7% UC, 82.3% control, p=0.06). This association was strongest in non-Jews (89.5% CD, 79.6% control, p=0.001).

The observation of an association between haplotype in GATA3 and CD shows that GATA3 variation contributes to CD pathogenesis through possible effects on Th1/Th2 dysregulation.

### Example 6

### Association Between a Haplotype of the GATA3 Gene and Inflammatory Bowel Disease: Subjects

A ease control panel was used from the IBD Center of Cedars-Sinai Medical Center, made up of 763 CD patients (314 Jewish), 351 UC patients (136 Jewish), and 254 controls (51 Jewish).

### Example 7

### Association Between a Haplotype of the GATA3 Gene and Inflammatory Bowel Disease: SNPs and Haplotypes

Seven SNPs in the GATA3 gene were selected from HapMap data to tag common Caucasian haplotypes and genotyped using the Illumina GoldenGate Assay. Haplotype blocks were constructed by Haploview 3.3, Individual haplotypes were obtained by PHASE 2.0.

### Example 8

### Association Between a Haplotype of the GATA3 Gene and Inflammatory Bowel Disease: Statistical Analysis

Analysis was done in the total sample, and then followed by Jews and non-Jews separately. Chi-square was used to test for association of haplotypes with IBD.

### Example 9

### Association Between a Haplotype of the GATA3 Gene and Inflammatory Bowel Disease: GATA3 Haplotypes and IBD

The most common haplotype from block 2, haplotype 1 (H1:1111) had a significantly higher carrier frequency in CD than in controls (CD: 88.7% vs control: 82.3%, p=0.008). H1 (block 2) carrier frequency was higher in UC than in controls (UC: 87.7% vs control: 82.3%, p=0.06). In the combined group (CD+UC), H1 (block 2) was significantly associated with IBD (IBD: 88.4% vs control: 82.3%, p=0.008).

### Example 10

### Association Between a Haplotype of the GATA3 Gene and Inflammatory Bowel Disease: Block 2 Haplotype 1 was Associated with IBD in Non-Jews

Analysis on Jews and non-Jews separately, haplotype 1 (block 2) was associated with the non-Jewish CD subjects and the non-Jewish IBD combined as well (in non-Jews, IBD: 88.3% vs control: 79.6%, p=0.001; CD: 89.5% vs control: 79.6%, p=0.001; UC: 85.6% vs control: 80.0%, p=0.1; in Jews, IBD: 88.7% vs control: 92.2%, p=0.45 CD: 87.6% vs control: 92.2%, p=0.34; UC: 91.2% vs control: 92.2%, p=0.83).

### Example 11

Association Between a Haplotype of the GATA3 Gene and Inflammatory Bowel Disease: Conclusions

The-observation of an association between a haplotype in GATA3 and IBD shows that GATA3 variation contributes to IBD pathogenesis through, possible effects on Th1/Th2 dysregulation.

### Example 12

### Variants in TL1A/TLR5/NOD2 Combine to Increase the Magnitude of Anti-Ompc Expression in Jewish Crohn's Disease Subjects

TNFSF15 (TL1A) Haplotype B (HB) is a CD risk haplotype in the Jewish population, with increased risk for anti-OmpC-positive Jews. Previously U was shown that the FcRgamma pathway (not TLR pathways) is crucial for inducing TL1A expression in monocytes and dendritic cells. NOD2 and TLR5 have previously been associated with CD and NOD2 has been associated with the expression of CD-associated microbial antibodies. Since interplay between the innate and adaptive immune systems likely leads to the response to microbial agents in CD, the inventors examined the relationship between genetic variation in these three genes and expression of various CD-associated anti-microbial antibodies.

5 TNFSF15 (TL1A) SNPs, 4 TLR5 SNPs and 3 CD-associated NOD2 SNPs were tested in 705 CD patients (255 Jews, 450 non-Jews). TNFSF1.5 SNPs are described herein as SEQ. ID. NO.: 29, SEQ. ID. NO.: 30, SEQ. ID. NO.: 31, SEQ. ID. NO.: 32 and SEQ. ID. NO.: 33. TLR5 SNPs are described herein as SEQ. ID. NO.: 34, SEQ. ID. NO.: 35, SEQ. ID. NO.: 36 and SEQ. ID. NO.: 37. Sera were analyzed for expression of and microbial antibodies by ELISA. Haplotypes of TNFSF15 (TL1A) and TLR5 were assigned by PHASEv2. Association was tested by chi-square and permutation of phenotypes. In Jews, the CD-associated TNFSF.15 HB was positively associated with anti-OmpC expression (42.3% of OmpC+ subjects were HB v 25.8% of OmpC−, p=0.006; mean anti-OmpC level for HB carriers was 23.9 v 15.5 for non-carriers, p=0.0032; contribution, to variance 2.1%). Also in Jews, TLR5 H2 was associated with anti-OmpC expression (71.4% of OmpC+ was H2 v 55.3%, p=0.O2; mean anti-OmpC level for H2 carriers was 21.4 v 15.2 for non-carriers, p=0.007, variance contribution: 3.5%). NOD2 by itself was not associated with anti-OmpC in Jews. However, the risk haplotypes from these 3 genes combined to increase anti-OmpC expression (41.9% with 1 genetic factor were OmpC+, 53.7%: with 2 were OmpC+, and 71.4% with 3, ptrend<0.0001: median level was 10.9 for 0, 19.4 for 1, 26.5 for 2, and 39.2 for 3, p trend<0.0001, 8.9% of variance explained). The OR for being anti-OmpC positive also increased with increasing number of genetic risk factors (3.5 for one factor, 5.6 for 2, and 12.1 for 3; p trend<0.0001). Furthermore, in Jews the sum by quartile of the expression of all antibodies also increased (mean quartile sum 9.1 for no genetic factors, 10.6 for 1, 10.9 for 2, and 11.8 for 3, p trend=0.007). There were no such relationships in the non-Jewish population.

In Jewish CD subjects, TNFSF15 (TL1A) HB, TLR5 H2, and NOD2 mutations are additive for increased expression of anti-OmpC. This observation supports the concepts that defects in both innate and adaptive immunity may be additive in CD and underscores the importance of the different populations in unraveling the complex interplay between these two immune systems in CD.

### Example 13

### TL1A/TLR5/NOD2—Methods

5 TL1A SNPs, 4 TLR5 SNPs and 3 CD-associated NOD2 SNPs were tested in 705 CD patients (255 Jews, 450 non-Jews). Sera were analyzed for expression of ASCA, anti-12, anti-OmpC and anti-CBir by ELISA. Haplotypes of TL1A and TLR5 were assigned by PHASE, The level of antibody was log-transformed before quantitative analysis. Quartile sum of the antibodies were calculated by assigning each antibody a quartile score according to its distribution (1,2,3,4) and summing 2.

### Example 14

### TL1A/TLR5/NOD2—Results

TL1A HB and TLR5 H2 were positively associated with anti-OmpC expression in Jews only. NOD2 by itself was not associated with anti-OmpC in Jews. However, the-risk haplotypes or variants from these 3 genes combined increased anti-OmpC expression. In Jews, the sum by quartile of the expression of all antibodies also increased with increasing number of genetic risk factors. The additive effect among TL1A, TLR5 and NOD2 in the expression of anti-OmpC and antibody quartile sum was not observed in the non-Jewish CD population. In Jewish CD subjects, TL1A HB, TLR5 H2, and NOD2 mutations are additive for increased expression of anti-OmpC.

### Example 15

### SIN(EFS) and Anti-Cbir1 Expression—Methods

654 Crohn's Disease subjects were used, with Crohn's Disease determined using standard criteria. The presence of anti-CBir1 antibody determined by ELISA. 7 SIN(EPS) SNPs were selected to tag major Caucasian haplotypes and genotyped by Illumina Golden Gate. 2 Non-Synonymous SNPs included R175W, rs2231805 and V100M, rs2231810. Association between SNPs and either CD or the presence of anti-CBir1 expression was tested by logistic regression. P-values empirically determined by permutation.

### Example 16

### SIN(EFS) and Anti-Cbir1 Expression—Results

The inventors tested the association of the SIN(EFS) gene with CD and expression of anti-CBir1 antibody. S1N(EFS) SNPs are described herein as SEQ. ID. NO.: 38, SEQ. ID. NO.: 39, SEQ. ID. NO.: 40, SEQ. ID. NO.: 41, SEQ. ID. NO.: 42, SEQ. ID. NO.: 43, SEQ. ID. NO.: 44, SEQ. ID. NO. 45, SEQ. ID. NO.: 46, SEQ. ID. NO.: 47 and SEQ. ID. NO.: 48. SIN(EFS) haplotype 2 was found to be associated with anti-CBir1 expression in human CD as was the non-synonymous SNP R175W variant (SEQ. ID. NO.: 45).

### Example 17

### BTLA and LIGHT Genetic Variation—Methods

Subjects studied included 763 CD patients: and 254 controls. Serum antibody detected by ELISA for Anti-I2 (IgA), Anti-OmpC (IgA), ASCA (IgG and IgA) and Anti-CBir1 (IgG). Clinical characteristics included disease location, such as small bowel only (L1), small bowel+colon (L3) and colon only (L2). Disease behavior was clinically diagnosed as fibrostenosis (B2), internal penetrating (B3), perianal penetrating (B3 p), UC-like (B1) and Small bowel surgery.

Statistical analyses included individual haplotypes obtained by PHASE and ordered by frequency in the entire sample. Mantel-Haenszel test was used for the association of haplotypes with CD and presence of antibody expression, with antibody level log transformed before analysis. T-test or ANOVA was used for the association between haplotypes and antibody level. Multiple logistic regression was used for disease behavior.

### Example 18

### BTLA Results—Table 1

TABLE 1
depicts results of BTLA variants associations with Crohn's Disease,
anti-I2 expression, and Small Bowel surgery phenotype. BTLA SNPs
are described herein as SEQ. ID. NO.: 49, SEQ. ID. NO.: 50,
SEQ. ID. NO.: 51, SEQ. ID. NO.: 52 and SEQ. ID. NO.: 53

| BTLA | CD | Anti-I2 | Surgery |
|---|---|---|---|
| Block1 H1 | P = 0.02 | P = 0.005 | NS |
| Block1 H3 | P < 0.0001 | NS | P = 0.008 |
| Block1 H4 | P < 0.0001 | NS | NS |

### Example 19

### LIGHT Results—Table 2

TABLE 2
depicts results of LIGHT variants associations with Crohn's Disease,
anti-I2 expression, and Fibrostenosis phenotype. LIGHT SNPs
are described herein as SEQ. ID. NO.: 54, SEQ. ID.
NO.: 55, SEQ. ID. NO.: 56 and SEQ. ID. NO.: 57.

| LIGHT | CD | Anti-I2 | Fibrostenosis |
|---|---|---|---|
| Block1 H3 | NS | P = 0.004 | P = 0.026 |
| Block2 H1 | NS | P = 0.008 | NS |

### Example 20

### BTLA and LIGHT Genetic Variation—Results

BTLA and LIGHT act as a molecular switch in modulating T cell activation. The inventors found BTLA block1 to be associated with CD. Specifically, BTLA block1 H1 was associated with anti-I2 expression in CD, BTLA block1 H3 was associated with small bowel surgery in CD (protective). LIGHT was found to be associated with anti-I2 expression, specifically LIGHT block 1 H3 and LIGHT block 2 H1. LIGHT Block 1 H3 was found to be associated with fibrostenotic disease in CD (protective).

### Example 21

### MAGI2—Subjects

Subjects and controls were recruited at Cedars-Sinai Medical Center following approval of the Cedars-Sinai Medical Center Institutional Review Board. 681 CD cases, 259 UC cases and 195 control subjects were included in the study. IBD phenotype was assigned using a combination of standard endoscopic, histological, and radiographic features (Mow, et al, Gastroenterology, 2004. 126(2); p. 414-24). Controls were included in the study that had no personal or family history of IBD. All study subjects were Caucasian.

### Example 22

### MAGI2—Selection of SNPs

The single nucleotide polymorphisms (SNPs) for genotyping were selected using data from the Caucasian data of the International ‘HapMap’ (The International HapMap Project, Nature, 2003. 426(6968); p. 789-96; Barrett, J. C., et al, Bioinformatics, 2005. 21(2): p. 263-5; Frazer, K. A., et al. Nature, 2007. 449(7164): p. 851-61) and through utilizing the “Tagger” software program (Massachussets Institute of Technology). SNPs that were shown to tag the major Caucasian haplotypes and that were also compatible with Illumina technology were genotyped. The inventors aimed to identify SNPs in linkage disequilibrium with all SNPs in the HapMap data with a minor allele frequency ≧5%.

### Example 23

### MAGI2—Genotyping

DNA was extracted from Epstein Barr virus transformed lymphoblastoid ceil lines using a standard technique of proteinase K digestion, organic extraction, and ethanol precipitation. The SNPs were genotyped using the validated oligonucleotide ligation assay, Illumina Golden Gate technology (Illumina, San Diego, Calif.). The inventors genotyped 113 SNPs across the MAGI2 gene in 681 CD cases, 259 UC cases and 195 control subjects. One MAGI2 SNP was excluded from the analyses as it failed to meet the criteria for Hardy-Weinberg equilibrium.

### Example 24

### MAGI2—IBD Serological Status

Blood samples for-serological analyses were drawn following informed consent. Sera were analyzed at Cedars-Sinai Medical Center for expression of antibodies to oligomannan (anti-Saccromyces Cerevisiae (ASCA) (both IgG and IgA), the Pseudomonas fluorescens-related protein (I2), Escherichia Coli outer membrane porin C (anti-OMPC) and CBir1 flagellin (anti-CBir1) in a blinded fashion by enzyme-linked immunosorbent, assay (ELISA), as previously described (Mow, W. S., et al., Gastroenterology, 2004, 126(2): p. 414-24; Targan, S. R., et al., Gastroenterology, 20D5. 128(7): p. 2020-8). Any antibody level determined as equal, to or more, than 2 standard deviations above the population mean was designated as positive. Seroactivity to microbial antigens is a quantitative-trait and so antibody level was assessed ‘across’ each genotype (e.g. homozygote for common allele versus heterozygote versus homozygote for rare allele) using linear regression.

### Example 25

### MAGI2—Statistical Analyses

Case control analyses with the Chi squared test (Haploview v4) were used to test for association with any given phenotype. All p values reported are two-tailed p values and are not corrected for multiple testing. The p values for association with any phenotype have been represented graphically by calculating the Logarithm of the inverted P value (1/P). The inventors used Haploview v4 to determine haplotype blocks using the Gabriel et al confidence interval method Gabriel, S. B., et al., Science, 2002. 296(5576): p. 2225-9) and association with IBD phenotypes and immunophenotypes with all haplotypes was determined, within Haploview using the Chi squared test. Association with quantitative values of the IBD serologies was calculated in PLINK (Harvard University) using linear regression. SNPs were excluded from the analysis if they failed to meet Hardy-Weinberg equilibrium (p≦0.01).

### Example 26

### MAGI2—Results: IBD, UC and CD and MAGI2

The association between the MAGI2 SNPs and haplotypes with IBD, CD and UC are listed in full in table 1. A three marker intron 2 MAGI2 haplotype (rs7785088, rs323149 and rs13246026) was protective against IBD susceptibility (IBD haplotype frequency 8.2% vs. control haplotype frequency 12.9%, OR 0.61 (95% CI 0.44-0.86), p=0.006). CD was the main contributor to this association (OR 0.57 (CI 0.40-0,8.1), (p=0.002) with a trend towards association in UC with this haplotype (p=0.11). CD was also associated with alleles in introns 2, 5, 6 and 20 (table 1.). An intron 6 SNP (rs2160322) was significantly associated with UC with the common C allele being the risk allele (UC 72.2% vs control allele frequency 63.6% (OR 1.49 (1.12-1.97), (p=0.006)). Two further SNPs (rs7788384 and rs2110871) and a 2 marker haplotype (rs7803276/rs7803705) from intron 6 were associated with UC susceptibility. Intron 20 (p=0.03) and intron 2 (p=0.02) SNPs were also associated with UC.

### Example 27

TABLE 3
Association between MAGI2 SNPs and haplotypes and IBD, UC and CD

| MAGI2 SNP | MAGI2 | ‘Risk’ | Gene location | | ‘Disease’ | Control | | 95% | P |
|---|---|---|---|---|---|---|---|---|---|
| or Haplotype | locus | allele | (intron etc) | Phenotype | allele Hz | allele Hz | OR | CI | value |
| rs7785088/ | 1 | GTC | Intron 2 | IBD | 8.2% | 12.9% | 0.61 | 0.44-0.86 | 0.006 |
| rs323149/ | | | | | | | | | |
| rs13246026 | | | | | | | | | |
| rs10242163/ | 2 | GGGC | Intron 2 | IBD | 3.5% | 5.9% | 0.59 | 0.36-0.96 | 0.04 |
| rs10808167/ | | | | | | | | | |
| rs1207881/ | | | | | | | | | |
| rs10264296 | | | | | | | | | |
| rs10260232 | 3 | T | Intron 5 | IBD | 55.9% | 50.3% | 1.25 | 1.01-1.56 | 0.05 |
| rs2160322 | 4 | C | Intron 6 | IBD | 70.3% | 63.6% | 1.35 | 1.08-1.70 | 0.009 |
| rs7788384 | 5 | G | Intron 6 | IBD | 61.7% | 56.2% | 1.26 | 1.01-1.57 | 0.05 |
| rs7785088/ | 1 | GTC | Intron 2 | CD | 7.7% | 12.9% | 0.57 | 0.40-0.81 | 0.002 |
| rs323149/ | | | | | | | | | |
| rs13246026 | | | | | | | | | |
| rs10242163/ | 2 | GGGC | Intron 2 | CD | 3.1% | 5.9% | 0.51 | 0.30-0.85 | 0.01 |
| rs10808167/ | | | | | | | | | |
| rs1207881/ | | | | | | | | | |
| rs10264296 | | | | | | | | | |
| rs1026023 | 3 | T | Intron 5 | CD | 56.1% | 50.3% | 1.27 | 1.01-1.58 | 0.04 |
| rs2160322 | 4 | C | Intron 6 | CD | 69.6% | 63.6% | 1.31 | 1.03-1.66 | 0.03 |
| rs3807736 | | T | Intron 20 | CD | 56.3% | 50.3% | 1.28 | 1.02-1.60 | 0.04 |
| rs7799287 | | C | Intron 2 | UC | 39.8% | 32.1% | 1.40 | 1.06-1.84 | 0.02 |
| rs17151725/ | | GC | Intron 2 | UC | 39.8% | 32.1% | 1.40 | 1.06-1.84 | 0.02 |
| rs7799287 | | | | | | | | | |
| rs7803276/ | 6 | GC | Intron 6 | UC | 58.0% | 50.2% | 1.37 | 1.05-1.78 | 0.02 |
| rs7803705 | | | | | | | | | |
| rs7803276/ | 6 | AG | Intron 6 | UC | 21.5% | 30.4% | 0.62 | 0.46-0.84 | 0.002 |
| rs7803705 | | | | | | | | | |
| rs2160322 | 4 | C | Intron 6 | UC | 72.2% | 63.6% | 1.49 | 1.12-1.97 | 0.006 |
| rs7788384 | 5 | G | Intron 6 | UC | 64.3% | 56.2% | 1.40 | 1.07-1.84 | 0.01 |
| rs2110871 | | C | Intron 6 | UC | 73.7% | 66.7% | 1.40 | 1.05-1.88 | 0.02 |
| rs7791394 | 7 | G | Intron 20 | UC | 57.5% | 50.0% | 1.35 | 1.04-1.76 | 0.03 |

### Example 28

### MAGI2—Results: IBD Serotype and MAGI2

A number of MAGI2 alleles were associated with CD immunophenotypes. Anti-CBir1 positive CD is associated with an intron 3 SNP (T allele of rs10239917) (P=0.0002) and a 2 marker haplotype constructed from this SNP (rs10239917) and rs11773635 produces both risk a haplotype (allele TA, P=0.0002) and a protective haplotype (allele CA, P=0.0001) for ant-CBir1 positive CD. IgG ASCA positive CD is associated with 2 separate haplotype blocks within intron 6. The GC rs7803705/rs7803276 haplotype is protective for IgG ASCA positive. CD (P=0.003) and the rs6951193/rs759332 haplotype demonstrates both risk alleles (AA, P=0.01) and protective alleles (GA, P=0.009) for IgG ASCA positive CD. Anti-OMPC positive CD is associated with a SNP in intron 3 (rs11773635, OR 1.56 (CI 1.20-2.04); P=0.0009) and no fewer than 4 separate SNPs in intron 9 (rs798285, P=0.007; rs798287, P=0.008; rs798292, P=0.01 and rs798279, P=0.02) as well as an intronic 4 SNP (rs725555, P=0.001).

A number of studies have demonstrated that increasing numbers of seropositivity for antibodies to microbial antigens are associated with a more severe course of disease in CD (Devlin, S. M., et al., Gastroenterology, 2007. 132(2): p. 576-86.). The T allele of rs10239917 (intron 3) is associated with anti-CBir1 and IgG ASCA positive CD (P=0.000038) when, compared to those not positive for both serotypes. Similarly the intron 3 two marker haplotype (rs10239917/rs11773635) shows both a risk (CC, P=0.0055) and protective haplotype (CA, P=0.0008) with anti-CBir1 and anti-OMPc positive CD.

### Example 29

TABLE 4
Association between MAGI2 SNPs and haplolypes and CD associated immunophenotype

| MAGI2 SNP | MAGI2 | Risk | Gene | | Disease | Control | | 95% | P |
|---|---|---|---|---|---|---|---|---|---|
| or Haplotype | locus | allele | location | Phenotype | allele Hz | allele Hz | OR | CI | value |
| rs7791394 | 7 | C | Intron 20 | anti- | 57.6% | 50.6% | 1.32 | 1.07-1.64 | 0.01 |
| | | | | ASCA + ve | | | | | |
| | | | | CD¹ | | | | | |
| rs38121 | | C | Intron 2 | IgG | 72.2% | 66.2% | 1.35 | 1.07-1.70 | 0.02 |
| | | | | ASCA + ve | | | | | |
| | | | | CD² | | | | | |
| rs10239917 | 8 | T | Intron 3 | IgG | 44.2% | 36.3% | 1.39 | 1.12-1.73 | 0.003 |
| | | | | ASCA + ve | | | | | |
| | | | | CD² | | | | | |
| rs319870 | | A | Intron 3 | IgG | 80.1% | 74.7% | 1.36 | 1.05-1.76 | 0.02 |
| | | | | ASCA + ve | | | | | |
| | | | | CD² | | | | | |
| rs17437602 | | T | Intron 3 | IgG | 70.1% | 64.4% | 1.30 | 1.03-1.63 | 0.03 |
| | | | | ASCA + ve | | | | | |
| | | | | CD² | | | | | |
| rs10239917/ | 8 | TA | Intron 3 | IgG | 44.2% | 36.3% | 1.39 | 1.12-1.73 | 0.003 |
| rs11773635 | | | | ASCA + ve | | | | | |
| | | | | CD² | | | | | |
| rs7803705 | 6 | A | Intron 6 | IgG | 74.2% | 66.7% | 1.43 | 1.13-1.82 | 0.003 |
| | | | | ASCA + ve | | | | | |
| | | | | CD² | | | | | |
| rs6951193 | 12 | A | Intron 6 | IgG | 72.2% | 65.8% | 1.35 | 1.07-1.71 | 0.01 |
| | | | | ASCA + ve | | | | | |
| | | | | CD² | | | | | |
| rs7803276 | 6 | A | Intron 6 | IgG | 50.7% | 44.2% | 1.30 | 1.05-1.61 | 0.02 |
| | | | | ASCA + ve | | | | | |
| | | | | CD² | | | | | |
| rs7803705/ | 6 | GC | Intron 6 | IgG | 25.7% | 33.3% | 0.69 | 0.55-0.88 | 0.003 |
| rs7803276 | | | | ASCA + ve | | | | | |
| | | | | CD² | | | | | |
| rs6951193/ | 9 | AA | Intron 6 | IgG | 35.5% | 29.0% | 1.35 | 1.07-1.70 | 0.010 |
| rs759332 | | | | ASCA + ve | | | | | |
| | | | | CD² | | | | | |
| rs6951193/ | 9 | GA | Intron 6 | IgG | 27.4% | 34.0% | 0.74 | 0.58-0.93 | 0.009 |
| rs759332 | | | | ASCA + ve | | | | | |
| | | | | CD² | | | | | |
| rs7791394 | 7 | C | Intron 20 | IgG | 59.4% | 50.6% | 1.42 | 1.15-1.77 | 0.001 |
| | | | | ASCA + ve | | | | | |
| | | | | CD² | | | | | |
| rs4727608 | | C | Intron 20 | IgG | 71.8% | 66.2% | 1.30 | 1.03-1.65 | 0.03 |
| | | | | ASCA + ve | | | | | |
| | | | | CD² | | | | | |
| rs3807728 | | G | Intron 20 | I2 + ve | 49.0% | 43.1% | 1.27 | 1.02-1.58 | 0.03 |
| | | | | CD³ | | | | | |
| rs10264296 | 2 | C | Intron 2 | anti- | 55.7% | 49.1% | 1.25 | 1.02-1.54 | 0.02 |
| | | | | CBir1 + ve | | | | | |
| | | | | CD⁴ | | | | | |
| rs10808167 | 2 | A | Intron 2 | anti- | 53.0% | 47.0% | 1.27 | 1.02-1.58 | 0.03 |
| | | | | CBir1 + ve | | | | | |
| | | | | CD⁴ | | | | | |
| rs10242163 | 2 | G | Intron 2 | anti- | 59.0% | 53.2% | 1.27 | 1.02-1.58 | 0.04 |
| | | | | CBir1 + ve | | | | | |
| | | | | CD⁴ | | | | | |
| rs10239917 | 8 | T | Intron 3 | anti- | 43.6% | 33.5% | 1.53 | 1.22-1.92 | 0.0002 |
| | | | | CBir1 + ve | | | | | |
| | | | | CD⁴ | | | | | |
| rs10239917/ | 8 | CA | Intron 3 | anti- | 35.9% | 46.2% | 0.65 | 0.52-0.82 | 0.0001 |
| rs11773635 | | | | CBir1 + ve | | | | | |
| | | | | CD⁴ | | | | | |
| rs10239917/ | 8 | TA | Intron 3 | anti- | 43.6% | 33.5% | 1.53 | 1.22-1.92 | 0.0002 |
| rs11773635 | | | | CBir1 + ve | | | | | |
| | | | | CD⁴ | | | | | |
| rs2110630 | | A | Intron 4 | anti- | 36.0% | 29.9% | 1.31 | 1.04-1.66 | 0.02 |
| | | | | CBir1 + ve | | | | | |
| | | | | CD⁴ | | | | | |
| rs1990577 | | A | Intron 5 | anti- | 49.1% | 43.0% | 1.28 | 1.03-1.59 | 0.03 |
| | | | | CBir1 + ve | | | | | |
| | | | | CD⁴ | | | | | |
| rs3735442 | 10 | A | Exon 6 | anti- | 34.7% | 28.7% | 1.32 | 1.04-1.67 | 0.02 |
| | | | (ns) | CBir1 + ve | | | | | |
| | | | | CD⁴ | | | | | |
| rs759334 | 11 | A | Intron 6 | anti- | 52.6% | 45.7% | 1.32 | 1.06-1.64 | 0.01 |
| | | | | CBir1 + ve | | | | | |
| | | | | CD⁴ | | | | | |
| rs6951193/ | 9 | AA | Intron 6 | anti- | 34.4% | 27.5% | 1.38 | 1.09-1.75 | 0.008 |
| rs759333 | | | | CBir1 + ve | | | | | |
| | | | | CD⁴ | | | | | |
| rs7801139 | | C | Iniron 7 | anti- | 30.0% | 24.0% | 1.36 | 1.06-1.74 | 0.02 |
| | | | | CBir1 + ve | | | | | |
| | | | | CD⁴ | | | | | |
| rs798292 | | A | Intron 9 | anti- | 79.3% | 74.6% | 1.30 | 1.01-1.69 | 0.05 |
| | | | | CBir1 + ve | | | | | |
| | | | | CD⁴ | | | | | |
| rs11773635 | 8 | C | Intron 3 | anti- | 25.1% | 17.6% | 1.56 | 1.20-2.04 | 0.0009 |
| | | | | OmpC + ve | | | | | |
| | | | | CD⁵ | | | | | |
| rs10239917/ | 8 | CC | Intron 3 | anti- | 25.1% | 17.6% | 1.55 | 1.20-2.03 | 0.0009 |
| rs11773635 | | | | OmpC + ve | | | | | |
| | | | | CD⁵ | | | | | |
| rs10239917/ | 8 | CA | Intron 3 | anti- | 36.4% | 42.0% | 0.79 | 0.64-0.99 | 0.04 |
| rs11773635 | | | | OmpC + ve | | | | | |
| | | | | CD⁵ | | | | | |
| rs725555 | | G | Intron 4 | anti- | 25.2% | 18.0% | 1.53 | 1.18-1.99 | 0.001 |
| | | | | OmpC + ve | | | | | |
| | | | | CD⁵ | | | | | |
| rs759334 | 11 | A | Intron 6 | anti- | 54.1% | 47.4% | 1.30 | 1.05-1.62 | 0.02 |
| | | | | OmpC + ve | | | | | |
| | | | | CD⁵ | | | | | |
| rs798285 | | C | Intron 9 | anti- | 45.9% | 38.6% | 1.35 | 1.08-1.68 | 0.007 |
| | | | | OmpC + ve | | | | | |
| | | | | CD⁵ | | | | | |
| rs798287 | | G | Intron 9 | anti- | 45.9% | 38.7% | 1.35 | 1.08-1.68 | 0.008 |
| | | | | OmpC + ve | | | | | |
| | | | | CD⁵ | | | | | |
| rs798292 | 13 | A | Intron 9 | anti- | 81.1% | 75.2% | 1.41 | 1.09-1.85 | 0.01 |
| | | | | OmpC + ve | | | | | |
| | | | | CD⁵ | | | | | |
| rs798279 | 14 | G | Intron 9 | anti- | 80.9% | 75.5% | 1.37 | 1.05-1.79 | 0.02 |
| | | | | OmpC + ve | | | | | |
| | | | | CD⁵ | | | | | |

### Example 30

### MAGI2—Results: Quantitative Data

Antibody levels were analyzed using linear regression and the positive associations are shown in table 5. Interestingly the T allele of rs10239917 in intron 3 is also associated with IgG ASCA level.

### Example 31

TABLE 5
Association between quantitative antibody levels and MAGI2 SNPs

| | MAGI2 | Associated | Gene | Antibody | P |
|---|---|---|---|---|---|
| MAGI2 SNP | ‘locus’ | allele | position | level | value |
| rs10239917 | 8 | T | Intron 3 | anti-IgA ASCA | 0.001 |
| rs319872 | | G | Intron 3 | anti-IgA ASCA | 0.034 |
| rs17436052 | 15 | G | Intron 4 | anti-IgA ASCA | 0.0003 |
| rs1030015 | 16 | G | Intron 4 | anti-IgA ASCA | 0.039 |
| rs1990577 | 17 | A | Intron 5 | anti-IgA ASCA | 0.013 |
| rs3735442 | 10 | A | Exon 6 | anti-IgA ASCA | 0.019 |
| rs6951193 | 12 | G | Intron 6 | anti-IgA ASCA | 0.004 |
| rs7791394 | 7 | T | Intron 20 | anti-IgA ASCA | 0.038 |
| rs17454991 | | T | Intron 2 | anti-IgG ASCA | 0.023 |
| rs10239917 | 8 | T | Intron 3 | anti-IgG ASCA | 0.0008 |
| rs17436052 | 15 | G | Intron 4 | anti-IgG ASCA | 0.0002 |
| rs1990577 | | A | Intron 5 | anti-IgG ASCA | 0.029 |
| rs7803705 | 6 | G | Intron 6 | anti-IgG ASCA | 0.011 |
| rs6951193 | 9 | G | Intron 6 | anti-IgG ASCA | 0.007 |
| rs759334 | 11 | A | Intron 6 | anti-IgG ASCA | 0.011 |
| rs798343 | | T | Intron 9 | anti-IgG ASCA | 0.044 |
| rs798279 | 14 | A | Intron 9 | anti-IgG ASCA | 0.050 |
| rs798292 | 13 | G | Intron 9 | anti-IgG ASCA | 0.041 |
| rs798356 | | T | Intron 10 | anti-IgG ASCA | 0.038 |
| rs2107992 | | G | Intron 1 | anti-I2 | 0.040 |
| rs17436052 | 15 | G | Intron 4 | anti-I2 | 0.007 |
| rs1030015 | 16 | G | Intron 4 | anti-I2 | 0.022 |
| rs1990577 | 17 | A | Intron 5 | anti-I2 | 0.038 |
| rs3807694 | | A | Intron 16 | anti-I2 | 0.035 |
| rs12668675 | | C | Intron 16 | anti-CBir1 | 0.032 |

Various embodiments of the invention are described above in the Description of Invention. While these descriptions directly describe the above embodiments, it is understood that those skilled in the art may conceive modifications and/or variations to the specific embodiments shown and described herein. Any such modifications or variations that fail within the purview of this description are intended to be included therein as well. Unless specifically noted, it is the intention of the inventor that the words and phrases in the specification and claims be given, the ordinary and accustomed meanings to those of ordinary skill in the applicable art(s).

The foregoing description of various embodiments of the invention known to the applicant at this time of filing the application has been presented and is intended for the purposes of illustration and description. The present, description is not intended to be exhaustive-nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying oat the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

1. A method for diagnosing susceptibility to Inflammatory Bowel Disease in an individual, comprising:
determining the presence or absence of a risk haplotype at the DR3 locus in the individual; and
diagnosing susceptibility to Inflammatory Bowel Disease in the individual based upon the presence of the risk haplotype at the DR3 locus.

2. The method of claim 1, wherein the risk haplotype at the DR3 locus comprises DR3 H2.

3. The method of claim 1, wherein the individual is non-Jewish.

4. The method of claim 1, wherein the risk haplotype at the DR3 locus comprises SEQ. ID. NO.: 7, SEQ. ID. NO.: 8, SEQ. ID. NO.: 9, SEQ. ID. NO.: 10, SEQ. ID. NO.: 11, SEQ. ID. NO.: 12 and/or SEQ. ID. NO.: 13.

5. The method of claim 1, wherein the Inflammatory Bowel Disease comprises Crohn's Disease and/or ulcerative colitis.

6. A method of determining a low probability of developing inflammatory Bowel Disease in an individual, relative to a healthy individual, comprising:
determining the presence or absence of DR3 H1 in the individual; and
diagnosing a low probability of developing Inflammatory Bowel Disease in the individual, relative to a healthy subject, based upon the presence of DR3 H1.

7. The method of claim 6, wherein the individual is non-Jewish.

8. The method of claim 6, wherein DR3 H1 comprises SEQ. ID. NO.: 7, SEQ. ID. NO.: 8, SEQ. ID. NO: 9, SEQ. ID. NO.: 10, SEQ. ID. NO.: 11, SEQ. ID. NO.: 12 and/or SEQ. ID. NO: 13.

9. A method, of determining a low probability of developing Crohn's Disease in an individual, relative to a healthy individual, comprising:
determining the presence or absence of a protective haplotype at the DR3 locus in the individual;
determining the presence or absence, of a protective haplotype at the TL1A locus in the individual; and
diagnosing a low probability of developing Crohn's Disease in the individual, relative to a healthy subject, based upon the presence of the protective haplotype at the DR3 locus and the presence of the protective haplotype at the DR3 locus.

10. The method of claim 9, wherein the protective haplotype at the DR3 locus comprises DR3 H1.

11. The method of claim 9, wherein the protective haplotype at the TL1A locus comprises TL1A H2.

12. The method of claim 9, wherein the protective haplotype at the DR3 locus comprises SEQ. ID. NO.: 14, SEQ. ID. NO.: 15, SEQ. ID. NO.: 16, SEQ. ID. NO.: 17, SEQ. ID. NO.: 18 and/or SEQ. ID. NO.: 19.

13. The method of claim 9, wherein the individual is-non-Jewish.

14. A method of diagnosing susceptibility to Inflammatory Bowel Disease in an individual, comprising:
determining the presence or absence of a risk haplotype at the GATA3 locus in the individual; and
diagnosing susceptibility to Inflammatory Bowel Disease in the individual based upon the presence of the risk haplotype at the GATA3 locus.

15. The method of claim 14, wherein the risk haplotype at the GATA3 locus comprises GATA3 Block 2 Haplotype 1.

16. The method of claim 14, wherein the Inflammatory Bowel Disease comprises Crohn's Disease and/or ulcerative colitis.

17. The method of claim 14, wherein the risk haplotype at the GATA3 locus comprises SEQ. ID. NO.: 22, SEQ. ID. NO.: 23, SEQ. ID. NO.: 24, SEQ. ID. NO.: 25, SEQ. ID. NO.: 26, SEQ. ID. NO.: 27 and/or SEQ. ID. NO.: 28.

18. A method of diagnosing Crohn's Disease in an individual, comprising:
determining the presence or absence of a risk haplotype at the GATA3 locus in the individual;
determining the presence or absence of Th1/Th2 dysregulation; and
diagnosing susceptibility to Crohn's Disease in the individual based upon the presence of the risk haplotype at the GATA3 locus and the presence of Th1/Th2 dysregulation.

19. The method of claim 18, wherein the risk haplotype at the GATA3 locus comprises GATA3 Block 2 Haplotype 1.

20. The method of claim 18, wherein the individual is non-Jewish.

21. The method of claim 18, wherein the risk haplotype at the GATA3 locus comprises SEQ. ID. NO.: 22, SEQ. ID. NO.: 23, SEQ. ID. NO.: 24, SEQ. ID. NO.: 25, SEQ. ID. NO.: 26, SEQ. ID. NO.: 27 and/or SEQ. ID. NO.: 28.

22. A method of diagnosing susceptibility to Crohn's Disease, comprising:
determining the presence or absence of one or more risk haplotypes at the TL1A locus, TLR5 locus and NOD2 locus;
determining the presence or absence of a high expression relative to a healthy subject of anti-OmpC expression; and
diagnosing susceptibility to Crohn's Disease in the individual based upon the presence of one or more risk haplotypes at the TL1A locus, TLR5 locus and NOD2 locus and the presence of high expression relative to a healthy subject of anti-OmpC expression.

23. The method of claim. 22, wherein one of the one or more risk haplotypes comprises TL1A Haplotype B.

24. The method of claim 22, wherein one of the one or more risk haplotypes comprises TLR5 Haplotype 2.

25. The method of claim 22, wherein the individual is Jewish.

26. The method of claim 22, wherein one of the one or more risk haplotypes comprises SEQ. ID. NO.: 29, SEQ. ID. NO.; 30, SEQ. ID. NO.: 31, SEQ. ID. NO.: 32 and/or SEQ. ID. NO.: 33.

27. The method of claim 22, wherein one of the one more risk haplotypes comprises SEQ. ID. NO.: 34, SEQ. ID. NO.: 35, SEQ. ID. NO.: 36 and/or SEQ. ID. NO.: 37.

28. A method of diagnosing susceptibility to a subtype of Crohn's Disease in an individual, comprising;
determining the presence or absence of at least one risk haplotype in the individual, selected from the group consisting of TL1A Haplotype B and TLR5 Haplotype 2, and
determining the presence or absence of a high expression relative to a healthy subject of anti-OmpC expression in the individual,
wherein the presence of one or more risk haplotypes and the presence of high expression relative to a healthy subject of anti-OmpC is diagnostic of susceptibility to the subtype of Crohn's Disease in the individual.

29. The method of claim 28, wherein the presence of two of said risk haplotypes presents a greater susceptibility than the presence of one or none of said risk haplotypes, and the presence of one of said risk haplotypes presents a greater susceptibility than the presence of none of said risk haplotypes but less than the presence of two of said risk haplotypes.

30. The method of claim 28, wherein the individual is Jewish.

31. A method of diagnosing susceptibility to a subtype of Crohn's Disease in an individual, comprising:
determining the presence or absence of one or more risk haplotypes at the SIN(EFS) locus;
determining the presence or absence of a high expression relative to a healthy subject of anti-Cbir1 expression; and
diagnosing susceptibility to the subtype of Crohn's Disease in the individual based upon the presence of one or more risk haplotypes at the SIN(EFS) locus and the presence of high expression relative to a healthy subject of anti-Cbir1 expression.

32. The method of claim 31, wherein one of the one or more risk haplotypes at the SIN(EFS) locus comprises SIN(EFS) haplotype 2.

33. The method of claim 31, wherein one of the one or more risk haplotypes at the SIN(EFS) locus comprises SEQ. ID. NO.: 38, SEQ. ID. NO.; 39, SEQ. ID. NO.: 40, SEQ. ID. NO.: 41, SEQ. ID. NO.; 42. SEQ. ID. NO.; 43, SEQ. ID. NO.: 44, SEQ. ID. NO.: 45, SEQ. ID. NO.: 46, SEQ. ID. NO.: 47 and/or SEQ. ID. NO.: 48.

34. A method of diagnosing susceptibility to a subtype of Crohn's Disease in an individual, comprising;
determining the presence or absence of one or more risk haplotypes at the BTLA locus;
determining the presence or absence of a high expression relative to a healthy subject of anti-I2 expression; and
diagnosing susceptibility to the subtype of Crohn's Disease in the individual based upon the presence of one or more risk haplotypes at the BTLA locus and the presence of high expression relative to a healthy subject of anti-I2 expression.

35. The method of claim 34, wherein one of the one or more risk haplotypes at the BTLA locus comprises BTLA Block 1 Haplotype 1.

36. The method of claim 34, wherein one of the one or more risk haplotypes at the BTLA locus comprises SEQ. ID. NO.: 49, SEQ. ID. NO.; 50, SEQ. ID. NO.: 51, SEQ. ID. NO.: 52 and/or SEQ. ID. NO.: 53.

37. A method of diagnosing susceptibility to a subtype of Crohn's Disease in an individual, comprising:
determining the presence or absence of one or more risk haplotypes at the LIGHT locus;
determining the presence or absence of a high expression relative to a healthy subject of anti-I2 expression; and.
diagnosing susceptibility to the subtype of Crohn's Disease, in the individual based upon the presence of one or more risk haplotypes at the LIGHT locus and the presence of high expression relative to a healthy subject of anti-I2 expression.

38. The method of claim 37, wherein one of the one or more risk haplotypes at the LIGHT locus comprises LIGHT Block 2 Haplotype 2.

39. The method of claim 37, wherein one of the one or more risk haplotypes at the LIGHT locus comprises SEQ. ID. NO.: 54, SEQ. ID. NO.: 55, SEQ. ID. NO.: 56 and/or SEQ. ID. NO.: 57.

40. A method of determining a low probability of developing a subtype of Crohn's Disease in an individual, comprising:
determining the presence or absence of one or more protective haplotypes at the BTLA locus; and
diagnosing a low probability of developing the subtype of Crohn's Disease in the individual, relative to a healthy individual, based upon the presence of one or more protective haplotypes at the BTLA locus.

41. The method of claim 40, wherein one of the one or more protective haplotypes at the BTLA locus comprises BTLA Block 1 Haplotype 3.

42. The method of claim 40, wherein the subtype of Crohn's Disease comprises a small bowel surgery phenotype.

43. The method of claim 40, wherein one of the one or more protective haplotypes at the BTLA locus comprises SEQ. ID. NO.: 49, SEQ. ID. NO.: 50, SEQ. ID. NO.: 51, SEQ. ID. NO.: 52 and/or SEQ. ID. NO.: 53.

44. A method of determining a low probability of developing a subtype of Crohn's Disease in an individual, comprising:
determining the presence or absence of one or more protective haplotypes at the LIGHT locus; and
diagnosing a low probability of developing the subtype of Crohn's Disease in the individual, relative to a healthy individual, based upon the presence of one or more protective haplotypes at the LIGHT locus.

45. The method of claim 44, wherein one of the one or more protective haplotypes at the LIGHT locus comprises LIGHT Block 1 Haplotype 3.

46. The method of claim 44, wherein the subtype of Crohn's Disease comprises a fibrostenotic phenotype.

47. The method of claim 44, wherein one of the one or more protective haplotypes at the LIGHT locus comprises SEQ. ID. NO.: 54, SEQ. ID. NO.: 55, SEQ. ID. NO.: 56 and/or SEQ. ID. NO.: 57.

48. A method of diagnosing susceptibility to a subtype of Inflammatory Bowel Disease in an individual, comprising:
determining the presence or absence of one or more risk haplotypes at the MAGI2 locus in the individual; and
diagnosing susceptibility to the subtype of Inflammatory Bowel Disease based upon the presence of one or more risk haplotypes at the MAGI2 locus in the individual.

49. The method of claim 48, wherein one of the one or more risk haplotypes at the MAGI2 locus comprises a variant listed in Table 3, Table 4 and/or Table 5 herein.

50. A method of determining a low probability of developing a subtype of Inflammatory Bowel Disease in an individual, comprising:
determining the presence or absence of one or more protective haplotypes at the MAGI2 locus; and
diagnosing a low probability of developing the subtype of Crohn's Disease in the individual, relative to a healthy individual, based upon, the presence of one or more protective haplotypes at the MAGI2 locus.

51. The method of claim 50, wherein one of the one or more protective haplotypes at the MAGI2 focus comprises a variant listed, in Table 3, Table 4 and/or Table 5 herein.

52. A method of diagnosing susceptibility to a subtype of Inflammatory Bowel Disease in an individual, comprising:
determining the presence of one or more risk variants at the DR3 locus, GATA3 locus, SIN(EFS) locus, BTLA locus, LIGHT locus and MAGI2 locus in the individual; and
diagnosing susceptibility to the subtype of Inflammatory Bowel Disease in the individual based upon the presence of one or more risk variants at the DR3 locus, GATA3 locus, SIN(EFS) locus, BTLA locus, LIGHT locus and MAGI2 locus.
